(19) United States
(12) Patent Application Publication
(10) Pub. No.: US 2019/0218492 A1
(43) Pub. Date: Jul. 18, 2019

(54) SELF REGULATING BIOREACTOR APPARATUS AND METHODS

(71) Applicant: YALE UNIVERSITY, New Haven, CT (US)

(72) Inventors: Alexander J. ENGLER, New Haven, CT (US); Laura E. NIKLASON, Greenwich, CT (US); Andrew V. LE, Branford, CT (US)

(73) Assignee: YALE UNIVERSITY, New Haven, CT (US)

(21) Appl. No.: 16/331,463

(22) PCT Filed: Sep. 11, 2017
(86) PCT No.: PCT/US2017/050910

§ 371 Date: Mar. 7, 2019

Related U.S. Application Data
(60) Provisional application No. 62/394,444, filed on Sep. 14, 2016.

(51) Int. Cl. C12M 3/00; C12M 1/04; C12M 1/06; C12M 1/00; C12M 1/34; C12M 1/36

(52) U.S. Cl. CPC C12M 21/08; C12M 23/24; C12M 27/02; C12M 29/10; C12M 41/34; C12M 41/48; C12M 29/24; C12M 41/14

(57) ABSTRACT

One aspect of the invention provides a device for quantifying and controlling oxygen concentration within a bioreactor containing a cell-containing sample that is actively consuming oxygen. The device includes: a bioreactor vessel adapted and configured to receive a cell-containing sample; a perfusion loop adapted and configured to circulate a perfusate from within the bioreactor vessel and back into the bioreactor vessel, the perfusion loop including a first pump; a gas exchanger including one or more gas exchange sources adapted and configured to add or remove gases from the perfusate; a sensor within the bioreactor adapted and configured to measure the dissolved oxygen concentration in the perfusate; and a controller programmed to control one or more parameters selected from the group consisting of the specified flow rate of the perfusate through the gas exchanger and the rate of gas exchange through the one or more gas exchange sources.

### CROSS-REFERENCE TO RELATED APPLICATIONS

This application claims priority to U.S. Provisional Patent Application Ser. No. 62/394,444, filed Sep. 14, 2016, the entire disclosure of which is incorporated herein by reference.

### STATEMENT REGARDING FEDERALLY SPONSORED RESEARCH OR DEVELOPMENT

This invention was made with government support under grant number 1U01HL111016-01 awarded by the National Heart, Lung and Blood Institutes and grant number 5T32GM086287-08 awarded by the National Institute of General Medical Sciences. The government has certain rights in the invention.

### BACKGROUND OF THE INVENTION

Bioreactors serve as vessels for in vitro or ex vivo study and growth of cellular and tissue systems. The pharmaceutical industry has spearheaded development of cellular bioreactors, enabling large-scale production of proteins and antibodies from individual cells. However, systems for culturing intact, functional organs are crude by comparison. Whole organ culture systems in the prior art provide little to no control over gas exchange and nutrient levels. Additionally, there exists very little understanding about how mass transfer affects the growth of complex tissues, and computational models that contemplate in vitro or ex vivo organ maintenance are either rudimentary or non-existent.

### BRIEF SUMMARY OF THE INVENTION

One aspect of the invention provides a device for quantifying and controlling oxygen concentration within a bioreactor containing a cell-containing sample that is actively consuming oxygen. The device includes: a bioreactor vessel adapted and configured to receive a cell-containing sample; a perfusion loop adapted and configured to circulate a perfusate from within the bioreactor vessel and back into the bioreactor vessel, the perfusion loop including a first pump; a gas exchanger including one or more gas exchange sources adapted and configured to add or remove gases from the perfusate; a sensor within the bioreactor adapted and configured to measure the dissolved oxygen concentration in the perfusate; and a controller programmed to control one or more parameters selected from the group consisting of the specified flow rate of the perfusate through the gas exchanger and the rate of gas exchange through the one or more gas exchange sources.

This aspect of the invention can have a variety of embodiments. The device can only exchanges gases through the one or more gas exchange sources and be otherwise substantially sealed off from the ambient atmosphere. The gas exchanger can be integrated in-line into the perfusion loop. The controller can be further programmed to modulate the specified flow rate of gas exchange through the one or more gas exchange sources. The perfusion loop can be adapted and configured to circulate a perfusate from within the bioreactor vessel, through the cell-containing sample and back into the bioreactor vessel. The gas exchange sources can be adapted and configured to introduce oxygen into or remove oxygen from the perfusate.

The controller can be further programmed to calculate an oxygen consumption rate for the cell-containing sample. The controller can be programmed to calculate an oxygen consumption rate for the cell-containing sample utilizing a differential equation relating: instantaneous oxygen consumption rate; dissolved oxygen concentration in the perfusate; and known system parameters derived for the specific device configuration.

The controller can be further programmed to maintain a steady oxygen concentration in the perfusate in the event of a change in oxygen concentration. The change in oxygen concentration in the perfusate can be due to a change in oxygen consumption. The controller can be programmed to maintain a steady oxygen concentration in the perfusate by: calculating an oxygen consumption rate for the cell-containing sample utilizing a differential equation relating: instantaneous oxygen consumption rate; dissolved oxygen concentration in the perfusate; and known system parameters derived for the specific device configuration; and altering the system parameters in order to maintain a steady oxygen concentration in the perfusate. The change in oxygen consumption can be due to cell proliferation, cell degradation or metabolic shift within the cell-containing sample.

The oxygen consumption rate can be determined without sealing the system from the one or more gas exchange sources. Oxygen consumption can be tracked continuously in real time. The sensor can be selected from the group consisting of: an optical dissolved oxygen probe and a dissolved oxygen electrode.

The cell-containing sample can be selected from the group consisting of: a cell culture, a tissue segment, a partial organ, a whole organ and an organ mimic. The cell culture can be a culture comprising at least one selected from the group consisting of: adherent cells, cells suspended in a fluid, cells suspended in a gel and a self-assembling cellular scaffold.

The cell-containing sample can include tissue from one or more organs selected from lung, heart, kidney, liver, vessel, trachea, skin, pancreas, bladder, cartilage and bone. The cell-containing sample can be derived from a source selected from the group consisting of murine, canine, ovine, porcine, bovine and primate sources. The cell-containing sample can be derived from a human.

The perfusate can include a phosphate-buffered saline solution. The perfusate can include a culture medium containing one or more cellular growth factors and/or one or more nutrients.

The one or more gas exchange sources can be hollow fiber supported membranes that are exposed to a gas source. The supported membranes can include one or more materials selected from the group consisting of polydimethylsiloxane, polymethylpentene, polyethersulfone and polysulfone.

The gas source can be an oxygen source. The gas source can include at least about 0.001% oxygen by volume.

The controller can be further programmed to collect oxygen concentration levels and flow rates about every 500 milliseconds to about every 1 hour. The main body of the bioreactor can include a vessel including one or more materials selected from the group consisting of stainless steels, borosilicates, platinum-cured silicones, polysulfones, fluoropolymers, polyethylenes and acrylics.

The perfusate within the bioreactor can be stirred.

The gas exchanger can be a gas exchange loop adapted and configured to circulate the perfusate in the bioreactor vessel through the one or more gas exchange sources and back into the bioreactor vessel. The gas exchange loop can further include a second pump that is controllable to operate at a specified fluid flow rate. The rate of gas exchange through the one or more gas exchange sources can be constant. The controller can be programmed to control the specified flow rate of the perfusate through the gas exchange loop. The controller can be programmed to control the gas flow rate through the one or more gas exchange sources.

The controller can be programmed to calculate an oxygen consumption rate for the cell-containing sample by: receiving a dissolved oxygen concentration C_{B} value from the sensor; measuring a flow rate F_{O} for the gas exchange loop and a flow rate Fₚ for the perfusion loop; and solving the differential equation Ċ_{B}=F_{O} (C_{O}−C_{B})−F_{P}(C_{B}−C_{L}), wherein: C_{O} is a concentration of oxygen leaving the gas exchange sources; and C_{L} is a concentration of oxygen leaving the cell-containing sample.

The controller can be programmed to calculate an oxygen consumption rate for the cell-containing sample by: receiving a dissolved oxygen concentration C_{B} value from the sensor; measuring a flow rate F_{O} for the gas exchange loop; and solving the equation C_{B}=S(F_{O})−\dot{Q}₀·τ(F_{O})/V for oxygen consumption rate \dot{Q}₀, wherein: S(F_{O}) is an experimentally-determined system saturation function of F_{O}; (F_{O}) is an experimentally-determined system time constant as a function of F_{O}; and V is a total amount of fluid volume in the bioreactor, perfusion loop, and gas exchange loop. The controller can be further programmed to calculate an estimated average single cell oxygen consumption rate

$$\frac{{Q}_{0}}{{N}_{0}}$$

for a tissue sample comprising an initial known number of cells N₀.

The controller can be programmed to maintain a steady oxygen concentration in the perfusate through self-regulation by: measuring oxygen concentration C_{B} from within the bioreactor; measuring a flow rate F_{O} for the gas exchange loop; solving the equation C_{B}=S(F_{O})−\dot{Q}₀·τ(F_{O})/V for oxygen consumption rate \dot{Q}₀, wherein: S(F_{O}) is an experimentally-determined system saturation function of F_{O}; τ(F_{O}) is an experimentally-determined system time constant as a function of F_{O}; and V is a total amount of fluid volume in the bioreactor, perfusion loop, and gas exchange loop; and adjusting F_{O} in order to maintain a steady C_{B} value.

Another aspect of the invention provides a method of non-invasively estimating changes in a number of cells within a cell-containing sample using a device as described herein. The method includes: measuring oxygen concentration C_{B} from within the bioreactor; measuring a flow rate F_{O} for the gas exchange loop; solving the equation C_{B}=S(F_{O})−\dot{Q}₀·τ(F_{O})/V for oxygen consumption rate \dot{Q}₀ at an initial condition in which the cell-containing sample has a known number of cells N₀, wherein: S(F_{O}) is an experimentally-determined system saturation function of F_{O}; τ(F_{O}) is an experimentally-determined system time constant as a function of F_{O}; and V is a total amount of fluid volume in the bioreactor, perfusion loop, and gas exchange loop; and solving the equation C_{B}=S(F_{O})−\dot{Q}ₙ·τ(F_{O})/V for oxygen consumption rate \dot{Q}ₙ for a later condition in which the cell-containing sample has an unknown number of cells Nₙ; and solving the equation

$${N}_{n} = \frac{{\dot{Q}}_{n} \cdot {N}_{0}}{{\dot{Q}}_{0}} .$$

The devices can further include at least one sensor for measuring the concentration of at least one compound in the perfusate selected from the group consisting of: glucose, lactate, glutamate, glutamine and ammonia.

Another aspect of the invention provides a method of non-invasively estimating metabolic activity in a cell-containing sample using a device as described herein. The method includes: measuring a change in glucose ΔGₙ and a change in lactate ΔLₙ in the perfusate over a period of time under an initial condition; solving the equation % A₀=(2−ΔLₙ/ΔGₙ)/2 to determine the portion of cells participating in aerobic metabolism % A₀ under the initial condition; and solving the equation \dot{Q}₀=\dot{Q}_{1A}*% A₀*N₀ for single cell aerobic oxygen consumption rate \dot{Q}_{1A} at the initial condition in which the cell-containing sample has a known number of cells N₀.

Another aspect of the invention provides a method of non-invasively estimating changes in a number of cells within a cell-containing sample using a device as described herein, wherein fewer than 100% of cells are participating in aerobic metabolism. The method includes: measuring a change in glucose ΔGₙ and a change in lactate ΔLₙ in the perfusate over a period of time under an initial condition; solving the equation % A₀=(2−ΔLₙ/ΔGₙ)/2 to determine the portion of cells participating in aerobic metabolism % A₀ under the initial condition; solving the equation \dot{Q}₀=\dot{Q}_{1A}*% A₀*N₀ for single cell aerobic oxygen consumption rate \dot{Q}_{1A} at the initial condition in which the cell-containing sample has a known number of cells N₀; and calculating a portion of cells participating in aerobic metabolism during a later condition by a further method comprising: measuring a change in glucose ΔGₙ and a change in lactate ΔLₙ in the perfusate over a period of time during a culture period; solving an equation % Aₙ=(2−ΔLₙ/ΔGₙ)/2 to determine a portion of cells participating in aerobic metabolism % Aₙ; and solving the equation

$$\frac{{\dot{Q}}_{0}}{\% {A}_{0} \cdot {N}_{0}} = \frac{{\dot{Q}}_{n}}{\% {A}_{n} \cdot {N}_{n}}$$

for Nₙ, an unknown number of cells in the cell-containing sample during the later condition.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1A is a schematic of the bioreactor apparatus of the invention according to an embodiment of the invention.

FIG. 1B is an image of the bioreactor apparatus of the invention according to an embodiment of the invention.

FIG. 1C is a simplified diagram of the bioreactor apparatus of the invention, outlining the various parameters that may be applied to Equation 1, the system's overall governing differential equation.

FIG. 2A is a diagram of the bioreactor apparatus of the invention lacking the cell-containing sample, which can be used to characterize the inherent gas exchange properties of the bioreactor as a function of the gas exchange flow rate.

FIGS. 2B-2D are graphs detailing different experimentally determined parameters of a bioreactor apparatus of the invention according to an embodiment of the invention. FIG. 2B demonstrates the determination of the hypoxic-to-ambient equilibration that was characterized by the time constant τ (measuring how quickly the system can respond) and saturation S (how much oxygen the system can hold). FIG. 2C illustrates the first-order response of time constant, τ, to the gas exchange flow rate F_{O}. FIG. 2D demonstrates that saturation S has no response to F_{O}. N=6 for each flow rate.

FIGS. 3A-3C illustrate the characterization of native rat lung oxygen consumption in a sealed system. FIG. 3A is a diagram of the “No O₂” experimental setup for characterizing the inherent oxygen consumption rate of native rat lungs, wherein the amount of oxygen allowed into the system is minimized. FIG. 3B is a lumped parameter model for gas exchange in the sealed bioreactor illustrated in FIG. 3A, with Equation 2 being the system's overall governing differential equation. FIG. 3C is a graph illustrating the raw dissolved oxygen versus time curves for the N=3 lungs tested. Lung #2 (dotted line) showed a local maximum at t=4 hours, caused by a stir plate malfunction, demonstrating the effect of stirring during the course of the culture.

FIGS. 4A-4D are graphs demonstrating validation of the derived mathematical models of the invention. FIG. 4A is a graph plotting dissolved oxygen versus time curves for each of four different F_{O} flow rates through the hollow fiber cartridge (HFC) gas exchange source. FIG. 4B illustrates the mathematical model predictions plotted against the actual equilibration values, calculated as the mean of the flattest 4 hour region after t=8 hours. N=3 for each flow rate. FIG. 4C is a graph plotting real-time oxygen consumption rate versus time curves, obtained by transforming the data reported in FIG. 4A using the mathematical models of the invention. FIG. 4D reports the oxygen consumption rates calculated from the equilibrium values reported in FIG. 4B.

FIGS. 5A-5R are images of rat lungs cultured in the bioreactor apparatus of the invention.

FIGS. 5A-5F are representative hematoxylin and eosin (H&E) stained rat lung images, 40× magnification, showing both distal and proximal airways from control lungs (FIG. 5A) fixed immediately post explant, lungs exposed to each of the four experimental F_{O} flow rates (FIGS. 5B-5E) and lungs deprived of oxygen flow (FIG. 5F). Scale bars are 50 μm.

FIGS. 5G-5L are representative proliferating cell nuclear antigen (PCNA) stained rat lung images of distal alveolar regions for all six experimental groups. Scale bars are 20 μm. FIGS. 5M-5R are representative terminal deoxynucleotidyl transferase dUTP Nick End Labeling (TUNEL) stained rat lung images of distal alveolar regions for all six experimental groups. Scale bars are 20 μm.

FIGS. 5S and 5T are graphs reporting quantification of PCNA and TUNEL respectively, for N=3 separate images, each of a distal alveolar region with greater than 100 nuclei, for each of N=3 distinct lungs per experimental group. Percentage indicates the proportion of positive cells per high powered field (HPF). Images contained 167±33 nuclei (mean+SD).

FIGS. 6A-6B are graphs reporting raw dissolved oxygen during a HBE (FIG. 6A) cell culture and a A549 (FIG. 6B) cell culture. Media changes at t=24, 48, and 72 hours result in transient periods of higher-than-normal oxygen readings due to sensor re-equilibration.

FIGS. 7A-7B are graphs reporting whole-organ oxygen consumption during a HBE (FIG. 7A) cell culture and an A549 (FIG. 7B) cell culture. Media changes at t=24, 48, and 72 hours result in transient periods of higher-than-normal oxygen readings due to sensor re-equilibration.

FIGS. 8A-8B are graphs reporting estimated cell number during a HBE (FIG. 8A) cell culture and a A549 (FIG. 8B) cell culture. Media changes at t=24, 48, and 72 hours result in transient periods of higher-than-normal oxygen readings due to sensor re-equilibration.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described. As used herein, each of the following terms has the meaning associated with it in this section.

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the term “bioreactor” refers to an apparatus in which a biological reaction or process is carried out.

As used herein, the term “hypoxic” refers to a concentration of dissolved oxygen less than about 13%, corresponding to a partial pressure of about 100 mmHg, the physiologic partial pressure of oxygen in the alveoli of the lung.

As used herein, the term “cell-containing sample” refers to a structure that contains one or more cells. Examples include tissue samples, whole organs, cellularized scaffolds, and the like.

As used herein, the term “cellular hypoxia” refers to a cellular response to exposure to a hypoxic environment, often resulting in apoptosis, or cellular death.

As used herein, the term “anaerobic metabolism” refers to the cellular consumption of glucose to produce two molecules of lactate, with the lactate remaining in dissolved in solution.

The ratio of lactate produced to glucose consumed will be 2:1.

As used herein, the term “aerobic metabolism” refers to the cellular consumption of glucose to produce two molecules of lactate, both of which will be consumed through the Krebs cycle in the presence of sufficient levels of oxygen. The ratio of lactate produced to glucose consumed will be 0:1.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

The following abbreviations are used herein:
ANOVA Analysis of variance
BSA Bovine serum albumin
DMEM Dulbecco's Modified Eagle Medium
DO Dissolved Oxygen
FBS Fetal Bovine Serum
H&E Hematoxylin and eosin
HFC Hollow fiber cartridge
PBS Phosphate buffered saline
PCNA Proliferating cell nuclear antigen
SLPM Standard liters per minute
TUNEL Terminal deoxynucleotidyl transferase dUTP Nick End Labeling

### DETAILED DESCRIPTION OF THE INVENTION

The level of dissolved oxygen within a bioreactor system is one of the most crucial factors to monitor and control. If the environment becomes too hypoxic, cells will no longer have sufficient oxygen to undergo aerobic metabolism, which can lead to cellular hypoxia, apoptosis, and loss of overall organ function. One of the largest obstacles to long-term whole organ culture is providing enough oxygen to meet the increasing—and difficult to measure—metabolic demands of the growing tissue. Hence, there exists an unmet need for bioreactor systems that can tune the levels of dissolved oxygen in the bioreactor, thereby providing physiologic levels of dissolved oxygen throughout the entirety of the culture period, regardless of metabolic demands and cell number.

In certain aspects, the invention provides a novel bioreactor apparatus capable of quantifying and controlling oxygen concentration within the bioreactor. In other aspects, the invention provides a novel bioreactor apparatus that can determine the oxygen consumption rate of a cell-containing sample contained therein, in a non-invasive and non-destructive manner. In still other aspects, the invention provides a novel bioreactor device that can self-regulate the internal oxygen concentration based on the determined oxygen consumption rate of the cell-containing sample contained therein. In yet other embodiments, the invention provides a non-invasive method of estimating cell proliferation or degradation within the bioreactor of the invention based on the change in oxygen consumption.

### Oxygen-Regulating Bioreactor Apparatus

Bioreactors are useful devices for culturing living organisms in a controlled environment and have been extensively used to grow large batches of cellular cultures. However, culturing living tissue cultures or whole organs in a bioreactor setup is more complicated than cellular cultures.

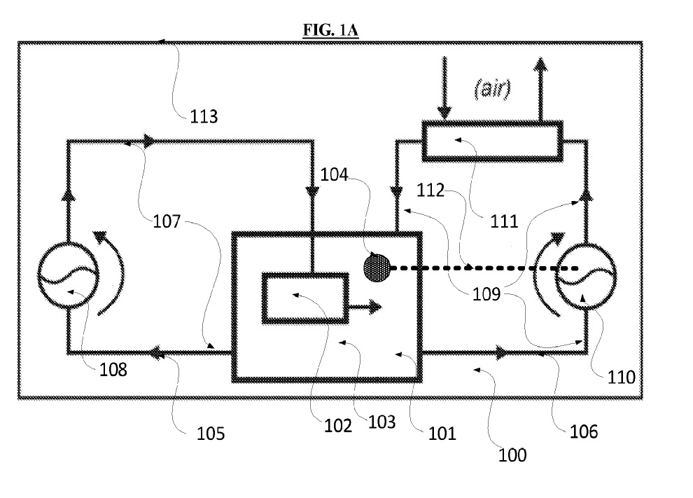

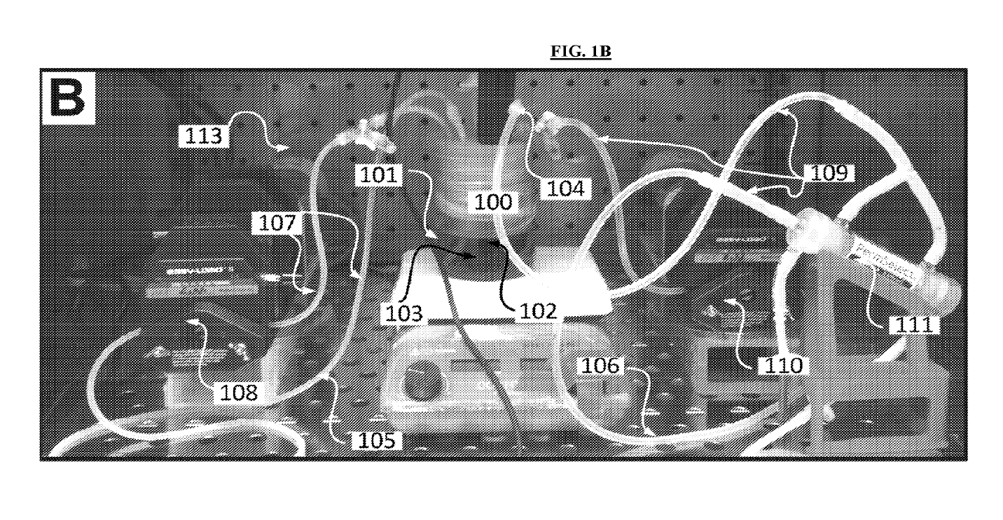

Referring now to FIGS. 1A-1B, one embodiment of the invention provides a novel apparatus comprising an oxygen regulating bioreactor 100. The bioreactor apparatus 100 can include a bioreactor vessel 101 which contains a cell-containing sample 102, a perfusate solution 103, and a dissolved oxygen concentration sensor 104. The bioreactor apparatus 100 can further comprise a perfusion loop 105 and a gas exchange loop 106, which are both in fluidic communication with the bioreactor vessel 101.

The perfusion loop 105 can include one or more lengths of perfusion tubing 107 and a perfusion pump 108. The perfusion loop 105 circulates the perfusate 103 from within the bioreactor vessel 101, optionally through a length of perfusion tubing 107, through a perfusion pump 108, optionally through a length of perfusion tubing 107, through the tissue sample 102 and back into the bioreactor vessel 101. In certain embodiments, the perfusion pump 108 is a pump with a controllable flow rate. In certain embodiments, the perfusion tubing 107 can be impermeable to oxygen, semipermeable to oxygen or permeable to oxygen. In other embodiments, the perfusion tubing 107 can have an oxygen permeability coefficient of about 5.0×10⁻¹⁰ (impermeable), about 2.00×10⁻⁹ (semipermeable), about 4.00×10⁻⁸ (highly permeable) or any permeability value in between.

The gas exchange loop 106 can include one or more lengths of gas exchange tubing 109, an gas exchange pump 110 and one or more gas exchange sources 111. In one embodiment, the gas exchange loop 106 circulates the perfusate 103 from the bioreactor vessel 101, optionally through a length of gas exchange tubing 109, through the gas exchange pump 110, optionally through a length of gas exchange tubing 109, through the one or more gas exchange sources 111, optionally through a length of gas exchange tubing 109, and back into the bioreactor vessel 101. In an alternate embodiment, the order of the gas exchange pump 110 and the one or more gas exchange sources 111 can be reversed or the perfusate 103 can flow through one or more gas exchange sources 111, through the gas exchange pump 110 and then through a second set of one or more gas exchange sources 111 before returning to the bioreactor vessel 101. The gas exchange pump 110 can be a pump with a controllable flow rate. In certain embodiments, the gas exchange tubing 109 can be impermeable to oxygen, semipermeable to oxygen or permeable to oxygen. In other embodiments, the gas exchange tubing 109 can have an oxygen permeability coefficient of about 5.0×10⁻¹⁰ (impermeable), about 2.00×10⁻⁹ (semipermeable), about 4.00×10⁻⁸ (highly permeable) or any permeability value in between. In certain embodiments, the gas exchange loop 106 modulates and regulates the oxygen concentration in the bioreactor apparatus 100. In other embodiments, the one or more gas exchange sources 111 are gas exchange sources which introduce oxygen into the system. The one or more gas exchange sources 111 can have between about 0.001% and about 100% oxygen by volume. In an alternative embodiment, the gas exchange sources 111 are hypoxic gas sources which extract oxygen from the system.

In certain embodiments, the bioreactor apparatus 100 further comprises a controller programmed to regulate the dissolved oxygen concentration within the perfusate 103. The controller can be a hardware and/or software device and can be in communication between the dissolved oxygen concentration sensor 104 and the gas exchange pump 110 along one or more communication links 112, wherein the flow rate of the gas exchange pump 110 can be modulated based on a reading from the dissolved oxygen concentration sensor 104. In certain embodiments, the controller can be a fully automated device that receives an input from the dissolved oxygen concentration sensor 104, determines whether to increase or decrease the flow rate of the gas exchange pump 110, and alter the flow rate accordingly without additional external input. In certain embodiments, the sensor 104 collects oxygen concentration values every 500 millisecond to about every 1 hour and alters the flow rate accordingly at the same time intervals. In other embodiments, the sensor 104 collects oxygen concentration values continuously and alters the flow rate continuously based on these measurements.

In certain embodiments, the bioreactor apparatus 100 only exchanges gases through the one or more gas exchange sources and is otherwise sealed off from the outside environment.

In some embodiments, the bioreactor apparatus 100 further comprises an incubator 113. The incubator 113 surrounds and contains one or more components of the bioreactor apparatus 100 selected from the group consisting of components 101-112. In a preferred embodiment, all of components 101-112 are contained within the incubator 113. The incubator 113 serves to provide a controlled environment for the bioreactor apparatus 100 to operate within. For example, the incubator 113 can be used to maintain a steady environmental temperature, humidity and atmospheric composition. In one embodiment, optimal incubator conditions for a tissue culture include a temperature of about 37° C., ambient atmospheric O₂ concentration, about 5% CO₂, and about 75% humidity. In certain embodiments, the incubator 113 can: maintain a temperature ranging from about 4° C. to about 42° C.; maintain an oxygen concentration ranging from 0% O₂ to 100% O₂; maintain a CO₂ concentration ranging from 0% to about 20%; maintain levels of humidity ranging from 0% to 100%; or any combination of conditions therein.

In some embodiments, the bioreactor vessel 100 is made of glass or any other material known in the art to be suitable for use in a bioreactor. These materials include, but are not limited to: stainless steels, borosilicates, platinum-cured silicones, polysulfones, fluoropolymers, polyethylenes, or acrylics.

In some embodiments, the dissolved oxygen concentration sensor 104 can be an optical dissolved oxygen probe or a dissolved oxygen electrode. In certain embodiments, the sensor 104 collects oxygen concentration values every 500 milliseconds to about every 1 hour. In other embodiments, the sensor 104 collects oxygen concentration values continuously.

In some embodiments, the perfusion tubing 107 and the gas exchange tubing 109 comprise biocompatible materials. In some embodiments, the perfusion tubing 107 and the gas exchange tubing 109 comprise one or more materials selected from silicone, BPT rubber, PVC plastic, Latex rubber, Gum rubber, EPDM rubber, TYGON® plastic, polypropylene, polyurethane rubber, fluorosilicone rubber, neoprene rubber, ethyl vinyl acetate plastic, polyethylene, polycarbonate, nylon, fluoropolymers, and the like. Tubing may be selected to be permeable, minimally permeable, or wholly impermeable to oxygen and other gasses.

In some embodiments, the perfusate 103 comprises a biocompatible buffered aqueous solution. In other embodiments, the perfusate 103 comprises a phosphate buffered saline solution (PBS). In yet other embodiments, the perfusate 103 further comprises nutrients and growth factors to promote growth and proliferation in the cell-containing sample. The perfusate 103 may be a common or uncommon cellular or tissue complete or partial culture medium, including but not limited to: DMEM, EMEM, CMEM, RPMI, F12, IMDM, M199, EGM, SAGM, BGJB, or any other commercially available culture medium. The perfusate 103 may also be a commercially available culture medium with one or more added growth factors, included but not limited to: amino acids, vitamins, antibiotics, antimycotics, steroid hormones, drugs, serum from any source, or any other commercially available culture additives. Finally, the perfusate 103 may be a custom or proprietary mix of nutrients and growth factors tailored to the specific tissue grown in the bioreactor 100.

In some embodiments, the perfusate 103 is stirred within the bioreactor vessel 101. Stirring speeds commonly used for tissue cultures can be used with the apparatus of the invention. In certain embodiments, the stirring speed can range from about 10 rpm to about 300 rpm, but preferably about 60 rpm.

In some embodiments, the one or more gas exchange sources 111 are hollow fiber supported membranes that are exposed to a gas source. In other embodiments, the membranes include one or more polymeric materials selected from the group selected from polydimethylsiloxane, polymethylpenetene, polyethersulfone and polysulfone. The membranes allow for gases to permeate into and out of the perfusate 103 without the perfusate 103 leaking out of the system. In certain embodiments, the gas source is air. In other embodiments, the gas source is a controlled gaseous mixture either comprising oxygen or not comprising oxygen. In yet other embodiments, the gas source comprises a mixture of oxygen, nitrogen, carbon dioxide and water vapor. In certain embodiments, the gas source is incubator air, preferably comprising ambient atmospheric O₂ concentration, about 5% CO₂, and about 75% humidity at about 37° C.

The apparatus can further include at least one sensor for measuring the concentration of nutrients and/or metabolic byproducts in the perfusate 103. For example, the apparatus can include a sensor that can determine changes in glucose concentration over time and/or changes in lactate/lactic acid concentration over time. In certain embodiments, the glucose sensor can be an optical sensor or an electrode sensor. In certain embodiments, the lactate sensor can be a lactate oxidase sensor or a lactate dehydrogenase sensor. In other embodiments, the lactate sensor can be a biosensor as described in Rathee, et al., Biochemistry and Biophysics Reports, Volume 5, March 2016, Pages 35-54. The sensor can also be a pH meter. In yet other embodiments, sensors may track glutamine, ammonia, or glutamate levels, or other intermediates or products of the Krebs cycle. In certain embodiments, these metabolic sensors may be integrated into the bioreactor, sampling from the perfusate between about once every 500 milliseconds to about once every one hour. In other embodiments, these metabolic sensors may be separate from the bioreactor, where culture medium is removed from the bioreactor and sampled at regular or irregular intervals ranging from every hour to every four days.

### Devices and Methods for Operating a Bioreactor Apparatus

The bioreactor apparatus described herein enables novel measurement and monitoring operation of the bioreactor without disturbing the reaction.

In some embodiments, the invention provides a bioreactor apparatus 100 wherein the oxygen consumption rate by the cell-containing sample 102 can be determined based on the rate of oxygen introduction and the dissolved oxygen concentration in the perfusate 103 by: measuring oxygen concentration C_{B} from within the bioreactor using dissolved oxygen concentration sensor 104; measuring a flow rate F_{O} for the gas exchange loop 106; and solving the equation C_{B}=S(F_{O})−\dot{Q}₀·τ(F_{O})/V for oxygen consumption rate \dot{Q}₀. S(F_{O}) can be an experimentally-determined system saturation function of F_{O}. τ(F_{O}) can be an experimentally-determined system time constant as a function of F_{O}; and V is a total amount of perfusate 103 volume in the bioreactor vessel 101, perfusion loop 105, and gas exchange loop 106.

In determining the oxygen consumption rate \dot{Q}₀ of the cell-containing sample 102, it is also possible to determine an average single cell oxygen consumption rate for a tissue sample comprising a known number of cells, N₀, wherein the average single cell consumption rate can be estimated as

$$\frac{{Q}_{0}}{{N}_{0}} .$$

The invention further provides a bioreactor apparatus 100 that is able to maintain a predetermined dissolved oxygen concentration in the perfusate 103 by: estimating oxygen consumption rate \dot{Q}₀ as described elsewhere herein and increasing or decreasing F_{O} in order to arrive at the predetermined C_{B} value.

In certain embodiments, the bioreactor apparatus 100 is able to maintain a stable dissolved oxygen concentration in the perfusate 103 while there are dynamic changes in oxygen consumption rate \dot{Q}₀ over a period of time by: continuously re-evaluating oxygen consumption rate \dot{Q}₀ at set time intervals; and continuously adjusting gas exchange flow rate (increase or decrease) F_{O} in order to maintain a steady dissolved oxygen concentration C_{B} in the perfusate 103.

In some embodiments, changes in oxygen consumption rate can be due to cell proliferation, cell degradation or metabolic shift within the cell-containing sample.

The bioreactor apparatus 100 of the invention is capable of measuring the oxygen consumption rate of the cell-containing sample without sealing off the system from the gas exchange source. As a result, oxygen consumption can be tracked continuously in real time and the dissolved oxygen concentration C_{B} in the perfusate 103 can be held at a steady concentration.

The invention additionally provides a method of non-invasively estimating cell proliferation within the cell-containing sample 102, using the bioreactor apparatus 100 of the invention, the method comprising: measuring oxygen concentration C_{B} from within the bioreactor using dissolved oxygen concentration sensor 104; measuring a flow rate F_{O} for the gas exchange loop 106; solving the equation C_{B}=S(F_{O})−\dot{Q}₀·τ(F_{O})/V for oxygen consumption rate \dot{Q}₀ at an initial condition in which the scaffold has a known number of cells N₀, wherein: S(F_{O}) is an experimentally-determined system saturation function of F_{O}; τ(F_{O}) is an experimentally-determined system time constant as a function of F_{O}; and V is a total amount of perfusate 103 volume in the bioreactor vessel 101, perfusion loop 105, and gas exchange loop 106; and solving the equation C_{B}=S(F_{O})−\dot{Q}ₙ·τ(F_{O})/V for oxygen consumption rate \dot{Q}ₙ for a later condition in which the scaffold has an unknown number of cells Nₙ; and solving the equation

$${N}_{n} = \frac{{\dot{Q}}_{n} \cdot {N}_{0}}{{\dot{Q}}_{0}} ,$$

using the derived values \dot{Q}₀ and \dot{Q}ₙ.

Although the description herein contains many embodiments, these should not be construed as limiting the scope of the invention but as merely providing illustrations of some of the presently preferred embodiments of the invention.

All references throughout this application (for example, patent documents including issued or granted patents or equivalents; patent application publications; and non-patent literature documents or other source material) are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application. In general, the terms and phrases used herein have their art-recognized meaning, which can be found by reference to standard texts, journal references and contexts known to those skilled in the art. Any preceding definitions are provided to clarify their specific use in the context of the invention.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

### Bioreactor Design

The bioreactor used for these studies is illustrated in FIG. 1A-B. All bioreactor components were obtained from COLE-PARMER® (Vernon Hills, Ill.) unless otherwise noted. The main body of the bioreactor was a 500 mL glass jar that was fitted with a silicone stopper, filled with 225 mL of culture medium, and stirred at 60 rpm. PHARMED® BPT tubing (Westlake, Ohio), size L/S 16, was inserted through the silicone stopper to enable the necessary connections to the lung, including “perfusion” and “gas exchange” loops, and to a pair of 0.22 m nylon air filters (WHATMAN®, GE Healthcare Life Sciences, Pittsburgh, Pa.). An optical dissolved oxygen probe (VERNIER®, Beaverton, Oreg.) extended into the bioreactor, with data from the probe collected with a LABQUEST® 2 interface every 5-10 seconds.

The perfusion loop consisted of 1.50 meters of PHARMED® silicone tubing and a MASTERFLEX® L/S roller pump (MASTERFLEX®, Vernon Hills, Ill.) to draw culture medium from the jar and perfuse it into the pulmonary arterial cannula. Culture medium was allowed to passively flow out of the pulmonary vein via the left ventricle and into the bioreactor jar. Parallel to this perfusion loop was an gas exchange loop, consisting of 1.50 total meters of PHARMED® silicone tubing fed through a second MASTERFLEX® L/S roller pump such that the speed of the two fluid loops could be independently controlled. Culture medium was drawn from the jar and pumped into a PDMS Hollow Fiber Cartridge (HFC, PERMSELECT®, Ann Arbor, Mich.), which then drained back into the bioreactor jar. This HFC consisted of 3200 thin-walled, hydrophobic fibers with an aggregate surface area of 2500 cm², roughly equivalent to the alveolar surface area of a 200 g SPRAGUE DAWLEY® rat. Incubator air (37° C., 5% CO₂, 75% humidity) was pumped through the fibers at 1.0 SLPM using an aquarium pump (JW Aquatic, Arlington, Tex.). The HFC was kept at a 300 orientation during culture using a 3D-printed stand to ensure it remained completely filled with fluid.

### Modeling Gas Exchange

To quantify and predict gas exchange within the bioreactor, a lumped parameter model was constructed as shown in FIG. 1C. The model contains three elements: (1) a “bioreactor” element, assumed to be a well-mixed fluid compartment with a concentration of dissolved oxygen C_{B}; (2) a “lung” element which consumes oxygen from the system; and (3) an “oxygenator” element which adds oxygen back into the system, consisting of all elements that introduce gasses into the system: the PHARMED® tubing, air filters, and hollow fiber cartridge. Fluid flows out of the bioreactor at oxygen concentration C_{B}, through the perfusion loop at flow rate F_{P}, and into the lung element. The oxygen concentration drops through the lung to a concentration C_{L}, and flows back into the bioreactor. Simultaneously and independently, fluid flows out of the bioreactor and through the gas exchange loop at flow rate F_{O}. The oxygen concentration rises through the oxygenator element to concentration C_{O}, and then flows back into the bioreactor. Since this is a “mixed-tank” lumped parameter model, a governing differential equation for the bioreactor oxygen concentration can be found by summing each product of flow rate and oxygen concentration in or out of the bioreactor (Eq. 1, FIG. 1C).

### Bioreactor Characterization

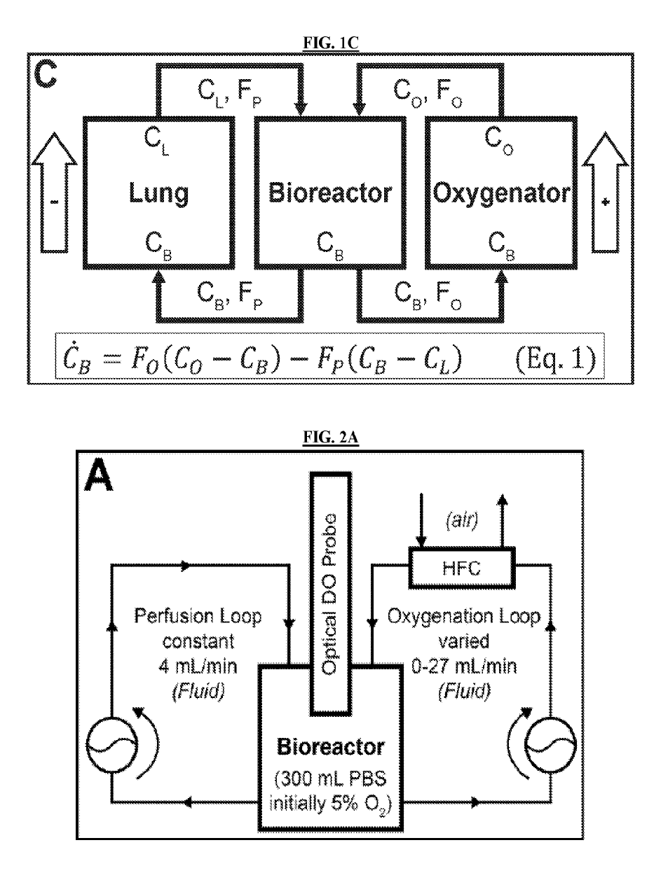

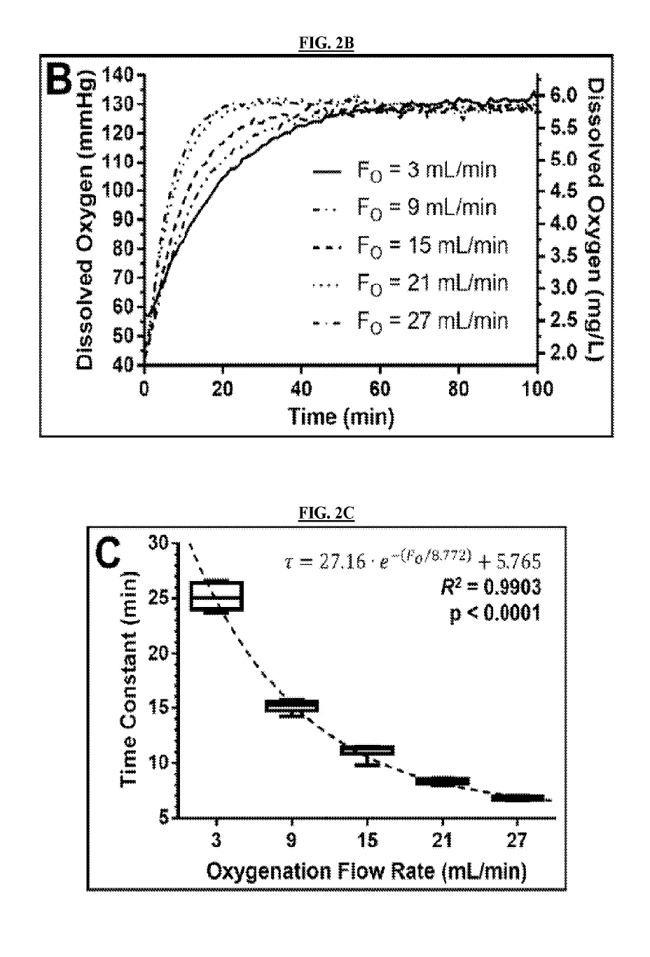

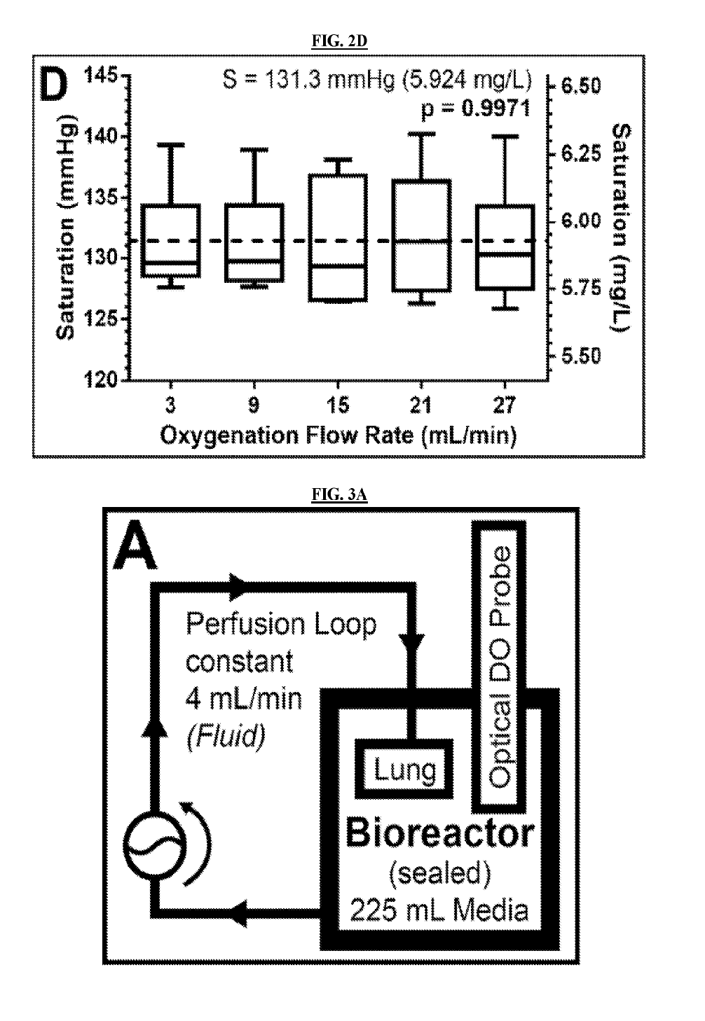

A schematic of the experiments performed for bioreactor characterization is shown in FIG. 2A. The bioreactor jar was filled with 300 mL of phosphate buffered saline (PBS) and was equilibrated to a hypoxic gas mixture of 5% O₂, 5% CO₂, 37° C., and 75% humidity. The cap, perfusion loop, and gas exchange loop with hollow fiber cartridge were assembled in a CARON® incubator (CARON®, Marietta, Ohio), at ambient O₂, 5% CO₂, 37° C., and 75% humidity. The air pump was placed inside the incubator and attached to the hollow fiber cartridge. The speed of the peristaltic pump for perfusion was fixed at 4 mL/min, and the speed of the peristaltic pump for gas exchange was set at 0, 3, 9, 15, 21, or 27 mL/min. The data acquisition interface was set to record dissolved oxygen concentration every five seconds for 100 minutes.

To initiate testing, the bioreactor jar was capped and quickly brought to the incubator. The hollow fiber cartridge, gas exchange line, and perfusion line were all primed with the hypoxic fluid (5% O₂). Data collection was begun concurrently with starting both peristaltic pumps. Data between t=5 minutes and t=100 minutes were fitted to a saturating exponential curve using the LABQUEST® interface, and the time constant and saturation point of the best fit curve were determined. For each non-zero HFC flow rate, N=6 experiments were performed. N=3 experiments were performed for the gas exchange flow rate of 0 mL/min. To determine if either the time constant or the saturation point were a function of gas exchange flow rate, a one-way ANOVA with Dunnett's multiple comparison test was used to test significance. Where significance was found, non-linear curve fitting in MATLAB® was utilized to determine the best fit relationship and quantify the correlation to gas exchange flow rate.

### Lung Harvest and Preparation

All animal work was performed in accordance with AAALAC guidelines and was approved by the Yale Institutional Animal Care and Use Committee (IACUC). Lungs were harvested from adult SPRAGUE DAWLEY® male rats weighing 308.8±11.1 grams (mean±SD). Briefly, rats were anesthetized with a mixture of 75 mg/kg of ketamine and 5 mg/kg xylazine, and the chest cavity was exposed following full deflation of the lungs. The heart was perfused with PBS containing heparin (100 U/mL, SIGMA®) and sodium nitroprusside (SNP, 10 μg/mL, SIGMA®) via the right ventricle. After 10 mL had been perfused, the heart, lungs, and trachea were removed en bloc.

Cannulae were inserted into the trachea and into the pulmonary artery (PA) via the right ventricle and attached with 4-0 polypropylene suture. Lungs were inflated with 10 mL of PBS with 1000 U/mL penicillin, 1000 μg/mL streptomycin (both from GIBCO®), 10 μg/mL amphotericin B, and 200 μg/mL gentamicin (both from GEMINI BIOPRODUCTS®). This solution was held in the airways while 120 mL of PBS/Heparin/SNP were perfused into the pulmonary artery via gravity-driven flow at a pressure head of 30 cm H₂O. Fluid was allowed to passively flow out the pulmonary vein via the left ventricle. After repeating this treatment with the antibiotics/antimycotics solution and final rinses with PBS and DMEM High Glucose (HG) complete medium, lungs were submerged first in 70% ethanol and next in PBS, then mounted in a bioreactor prepared with 225 mL of DMEM HG complete medium with 10% FBS, 100 U/mL penicillin, 100 μg/mL streptomycin, 3 μg/mL amphotericin B, and 50 μg/mL gentamicin. The total preparation time was noted as the interval between the initiation of heart perfusion and the initiation of data collection inside the bioreactor.

### Lung Oxygen Consumption Characterization

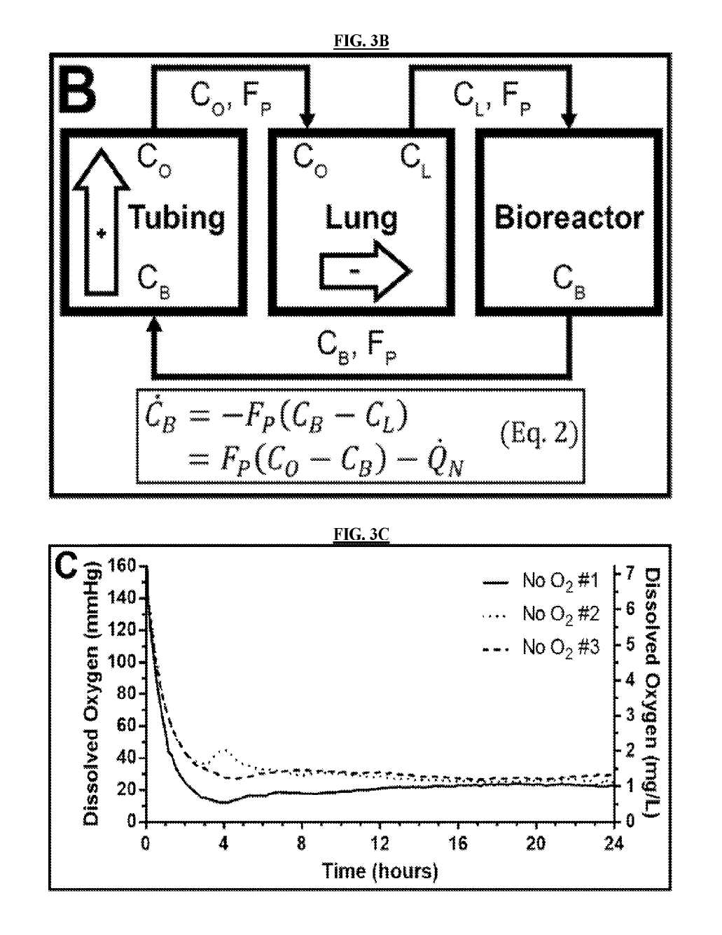

To characterize the inherent oxygen consumption characteristics of native lungs, lungs prepared as described above were placed into a sealed bioreactor with all routes of oxygen entry minimized or eliminated, as shown in FIG. 3A. The gas exchange loop with HFC was removed for these experiments, air filters were capped, and all silicone tubing replaced with low-oxygen-permeable PHARMED® tubing. A mathematical model for the sealed bioreactor is outlined in FIG. 3B, with Equation 2 as the governing differential equation. The oxygen probe data acquisition interface was set to record dissolved oxygen concentration every ten seconds for 24 hours of culture. The pulmonary artery perfusion speed was set to 4 mL/min for each culture period. N=3 total experiments were performed.

The initial whole-lung oxygen consumption rate was calculated as the slope of a linear regression of the dissolved oxygen vs time, between pO₂=120 and 80 mmHg. The equilibration point was calculated as the mean of the data between t=16 and 20 hours. The final oxygen consumption rate was calculated from the equilibration point by solving Equation 2 for the steady state condition. All regressions and calculations were performed in MATLAB.

### Solving the Mathematical Model

Results from the bioreactor characterization and the lung oxygen consumption characterization were incorporated into the mathematical model's differential equation (Equation 1) to determine a solution dependent upon only experimental parameters and experimentally-determined constants.

The governing differential equation for the model outlined in FIG. 1C is Ċ_{B}=F_{O} (C_{O}−C_{B})−F_{P}(C_{B}−C_{L}) (Equation 1). There are two unknowns: C_{O}, the concentration of oxygen leaving the oxygenator element, and C_{L}, the concentration of oxygen leaving the lung. C_{B}, the concentration of oxygen in the bioreactor, is directly measured, while the two flow rates are user-defined parameters for the HFC gas exchange loop (F_{O}) and for the pulmonary artery perfusion loop (F_{P}). To incorporate the results of bioreactor characterization testing, the expression F_{O}(C_{O}−C_{B}) can be replaced with the expression (S(F_{O})−C_{B})·V/τ(F_{O}), where S(F_{O}) is the experimentally-determined system saturation as a function of the gas exchange flow rate, τ(F_{O}) is the experimentally-determined system time constant as a function of the gas exchange flow rate, and V is the total amount of fluid volume in the system. Fick's Principle was utilized—in short, that (C_{B}−C_{L})=Q/F_{P}, where \dot{Q} is the experimentally-determined oxygen consumption rate for a native rat lung- and the expression F_{P} (C_{B}−C_{L}) was replaced with \dot{Q}. By setting Ċ_{B} to zero (e.g. by assuming the bioreactor oxygen concentration is not changing), the steady-state concentration of oxygen can be solved for within the bioreactor by rearranging the equation to isolate C_{B}:

C_{B}=S(F_{O})−\dot{Q}·τ(F_{O})/V (Equation 3)

This equation is now dependent only upon the gas exchange flow rate, which is a known experimental parameter, as well as experimentally determined mathematical relationships. Equation 3 therefore represents a quantification of the inherent, user-controlled gas transfer characteristics of the oxygenator-containing bioreactor system.

### Model Validation

To validate the model, lungs were cultured in the full bioreactor system over a range of HFC gas exchange flow rates: 4, 8, 16, or 32 mL/min. The perfusion flow rate was held constant at 4 mL/min. The data acquisition interface was set to record dissolved oxygen concentration every ten seconds for 24 hours of culture. N=3 independent experiments were performed at each of the four gas exchange flow rates, for a total of 12 native lung cultures. The equilibration point was determined as the mean of the flattest 4 hour region after t=8 hours. By plugging the raw dissolved oxygen data (C_{B}) and the HFC flow rate (F_{O}) into the mathematical model, the dissolved oxygen data were then transformed into an expression for the whole-lung oxygen consumption rate (\dot{Q}) vs time.

### Lung Takedown, Histology, and Immunofluorescence

After 24 hours of culture, glucose and lactate measurements of the culture medium were taken with a GLUCCELL® glucose meter (CESCO BioProducts, Atlanta, Ga.) and an I-STAT® cartridge (CG4+, ABAXIS®, Union City, Calif.) respectively. Glucose consumption and lactate production were calculated by subtracting baseline values of the culture medium from the post-culture values. The accessory, right caudal, and right medial lobes of the lung were tied off, removed, and weighed before snap-freezing. The left lobe and right cranial lobes were inflation-fixed for five minutes in 10% neutral buffered formalin (NBF) under 15 cmH₂O of intra-tracheal pressure, rocked for an additional three hours in NBF, weighed, and then paraffin-embedded and sectioned. Three control lungs were also prepared by fixation immediately after explant. Routine histology (hematoxylin and eosin, H&E) was performed for each lung, as well as immunofluorescence for PCNA (Proliferating Cell Nuclear Antigen) and TUNEL (Terminal deoxynucleotidyl transferase dUTP Nick End Labeling) to investigate proliferation and apoptosis, respectively.

For PCNA staining, 5 μm sections were rehydrated with a decreasing ethanol gradient, taken through antigen retrieval with citrate buffer (10 mM citric acid, 0.05% Tween 20, pH 6.0), permeabilized in PBS with 0.2% Triton X-100 for 15 minutes, and blocked in PBS with 0.75% glycine and 5% bovine serum albumin (BSA) for 60 minutes at room temperature. Blocked sections were incubated overnight at 4° C. with a primary PCNA antibody diluted in blocking buffer (Mouse, ABCAM®, 1:1000 dilution), and a secondary antibody was applied at a 1:500 dilution (Goat anti-Mouse, IgG ALEXA FLUOR® 555, INVITROGEN®). Sections were rinsed with PBS, co-stained with DAPI (BIOTIUM®, 1:1000 in Millipore ddH₂O), mounted with FLUOROMOUNT™ (SIGMA®), and imaged.

For TUNEL staining, samples were permeabilized following rehydration using a solution of 0.1% TRITON® X-100 and 0.1% sodium citrate, and incubated at room temperature for 8 minutes. 100 μL of an enzyme solution was added to 900 μL of a label solution from a TUNEL kit (ROCHE APPLIED SCIENCE®, Indianapolis, Ind.) and kept on ice. 50 μL of the TUNEL reaction mixture were added to each slide, after which sections were covered with a coverslip and incubated for 60 minutes at 37° C. in a humidified atmosphere in the dark. Samples were then rinsed with PBS, co-stained with DAPI, mounted, and imaged.

For histological quantification, three 40× images of distal alveolar regions with greater than 100 nuclei were taken for each PCNA and TUNEL slide, and the percentage of positive cells per high-powered field was calculated. N=3 separate images were taken for each N=3 distinct lungs per experimental group. A one-way ANOVA with Dunnett's multiple comparison test was used to test significance between experimental groups, with statistical significance characterized by p<0.05. All statistics were performed using the GRAPHPAD PRISM® statistical analysis software.

### Total DNA Analysis

Each accessory lobe was lyophilized overnight then digested in 1 mL of papain solution (1200 U papain [SIGMA-ALDRICH®], 40 mL PBS, 400 μL EDTA 0.5 M pH 8.0, 35.2 mg cysteine HCl, and 320 mg sodium acetate) for every 10 mg of dry weight. Samples were digested at 65° C. for 3 days and vortexed every 12 hours. Digested samples were diluted 1:100 in 1×TE buffer (1 mL 1M Tris-HCl pH 7.4, 0.2 mL EDTA, 99 mL Millipore ddH2O) to a total volume of 400 μL. 100 μL of each sample were placed in three wells in a 96-well plate along with 100 μL of PICOGREEN® fluorophore (LIFE TECHNOLOGIES®, Waltham, Mass.). The 96 well plate was placed in a fluorometer plate reader (SYNERGY® HT Multi-Detection Microplate Reader, BIOTEK, Winooski, Vt.) with excitation at 485 nm and emission at 535 nm. The estimated cell count in each sample was found by assuming 7 pg of DNA per cell. The estimated whole lung cell count was found by dividing the estimated sample cell count by the wet weight of each accessory lobe and multiplying by the combined weight of all five lobes.

### Variable Correlations

To investigate correlations between experimental variables, data from the 12 model validation lungs were analyzed in GRAPHPAD PRISM®. Independent variables tested were the HFC flow rate, rat weight, total lung weight, and preparation time. Dependent variables tested were the equilibrium pO₂; the whole-lung oxygen consumption rate at the equilibrium pO₂; Δ glucose and Δ lactate during culture; the ratio between lactate production and glucose consumption, Y_{l/g}; the estimated whole lung cell count; the single cell oxygen consumption rate, glucose consumption, and lactate production; and the percentage of cells positive for either PCNA or TUNEL. One-way ANOVAs with Dunnett's multiple comparison test were used to test significance against the HFC flow rate, with statistical significance characterized by p<0.05. Linear regressions were performed against rat weight, total lung weight, or prep time, and the correlation R² between the data was found. An F test was performed to test if the slope of the regression was significantly different than zero, with significance characterized by p<0.05.

### Example 1: Bioreactor Characterization

A simplified diagram of the experimental setup for bioreactor characterization is shown in FIG. 2A. Representative curves for each gas exchange flow rate are shown in FIG. 2B. Gas exchange curves were fitted to the equation for a saturating exponential to determine the time constant and the saturation point. The time constant for the system showed an exponential dependence upon the gas exchange flow rate (FIG. 2C), obeying the relationship τ(F_{O})=A·exp(−F_{O}/T)+B, where A, B, and T are the constants determined through the non-linear curve fitting in MATLAB. The time constant data fit the above equation with an R² of 0.9903, suggesting a first-order relationship between the gas exchange flow rate and the time constant, as expected (p<0.0001). The time constant for equilibration without a hollow fiber cartridge (analogous to an gas exchange flow rate of 0 mL/min) was 84.5±10.6 min.

FIG. 2D shows the dependence of the saturation point on the gas exchange flow rate, demonstrating that the saturation point of the system has no statistically significant dependence on gas exchange flow rate (p=0.9971). The mean of all values of the saturation point of oxygen in the bioreactor system was S=5.924 mg/L (131.3 mmHg). In the experiments performed without a hollow fiber cartridge, the saturation value was 5.877±0.02 mg/L (130.3±0.5 mmHg). This result suggests that the addition of the hollow fiber cartridge does not significantly affect the total amount of dissolved oxygen that the system can hold.

### Example 2: Lung Oxygen Consumption and DNA Analysis for Cell Number

A diagram of the experimental setup for lung oxygen consumption characterization is shown in FIG. 3A, with a lumped parameter model of the simplified system shown in FIG. 3B. The dissolved oxygen curves for the three lungs that were deprived of oxygen are shown in FIG. 3C, with oxygen consumption rates and equilibration values listed in Table 1. The preparation time (time from cardiac perfusion to data collection inside the bioreactor) for these three lungs was 33±5 min.

For lungs cultured without exogenous sources of oxygen, three different phases of oxygen consumption behavior can be seen. The first phase is a linear decrease in the levels of dissolved oxygen, indicating that the overall rate of oxygen consumption is fairly constant. The initial oxygen consumption rate was defined as the slope centered around 100 mmHg, and was equal to 1.432±0.223 mmHg/min (0.0676 mg/L/min, 0.475 μmol/min). The second phase consists of a shift towards exponential-like behavior at an inflection point occurring around 40-60 mmHg. This could mark a shift in the metabolic state of the lung, with possible upregulation of anaerobic metabolism over aerobic metabolism. Finally, there is an equilibration phase where the slope of the curve is approximately zero. This equilibration at 24.15±1.82 mmHg could indicate that the lungs are beginning to utilize oxygen at the same rate that oxygen is entering passively through the PHARMED® tubing in the bioreactor system. In equation 2 (FIG. 3B), this is equivalent to setting the left side of the equation to zero and rearranging to obtain: \dot{Q}_{N}=F_{P}(C_{O}−C_{B}). Given the PHARMED® tubing's oxygen permeability coefficient, surface area, and thickness, the oxygen consumption rate at the equilibration point was 0.0751±0.0013 mmHg/min (0.025 μmol/min), or approximately 5% of the initial oxygen consumption rate.

Taken together, some conclusions may be drawn about the oxygen utilization behavior of native rat lungs. First, given sufficient levels of dissolved oxygen, lungs will consume oxygen at a fixed rate, proportional to the percentage of cells that are able to participate in aerobic metabolism. Second, when levels of dissolved oxygen drop too low and are maintained low, the lung will slow its consumption rate of oxygen, possibly due to a shift from largely aerobic to largely anaerobic metabolism, or possibly due to the ischemia/necrosis of a percentage of cells in the lung. For the lungs tested here, this threshold value appears to range between 40 and 60 mmHg. Third, the lung is able to partially recover from periods of minimal or zero oxygen consumption, as evidenced by the equilibration of dissolved oxygen levels following a local minimum and subsequent rise.

The estimated whole lung cell count was determined through the DNA assay as described above, with the lungs containing 390±103 million cells. The mean single cell oxygen consumption rate was calculated as 1.301×10¹⁵ mol/min/cell, which is in good agreement with previously reported values for lung epithelium at 1.12×10¹⁵ mol/min/cell. This implies that the preparation time for the lungs is short enough to enable a majority of cells to participate in aerobic metabolism.

TABLE 1

| | Initial O₂ | | Final O₂ | Estimated | Estimated Single |
|---|---|---|---|---|---|
| | Consumption | | Consumption | Whole | Cell O₂ |
| | Rate | Equilibration | Rate | Lung Cell | Consumption |
| | (mmHg/min | Point (mmHg | (mmHg/min | Count | Rate |
| Lung # | [mg/L/min]) | [mg/L]) | [mg/L/min]) | (millions) | (mol/min/cell) |
| 1 | 1.688 (0.0797) | 22.29 (1.053) | 0.0764 | 299 | 1.877E−15 |
| | | | (0.00344) | | |
| 2 | 1.332 (0.0629) | 24.23 (1.145) | 0.0750 | 502 | 0.882E−15 |
| | | | (0.00338) | | |
| 3 | 1.276 (0.0603) | 25.93 (1.225) | 0.0738 | 370 | 1.144E−15 |
| | | | (0.00333) | | |
| Mean ± | 1.432 ± 0.223 | 24.15 ± 1.82 | 0.0751 ± | 390 ± 103 | 1.301E−15 ± |
| SD | (0.0676 ± | (1.141 ± | 0.0013 | | 0.515E−15 |
| | 0.0105) | 0.086) | (0.00338 ± | | |
| | | | 0.00005) | | |

### Example 3: Model Validation

The bioreactor and lung characterization data were incorporated into Equation 3 in order to obtain a quantitative relationship between the steady state concentration of oxygen in the bioreactor and the gas exchange flow rate. Experimentally-determined constants for τ(F_{O}) (FIG. 2C) and S(F_{O}) (FIG. 2D) were combined with the value for \dot{Q}_{N}, the mean whole-lung oxygen consumption rate from Table 1 and FIG. 3C. This results in:

C_{B}=S−(\dot{Q}_{N}/V)·(A·exp(−F_{O}/T)+B)

C_{B}=(5.924 mg/L)−(0.0676 mg/L/min)·((27.16 min)·exp(−F_{O}/(8.772 mL/min))+(5.765 min)) (Equation 3)

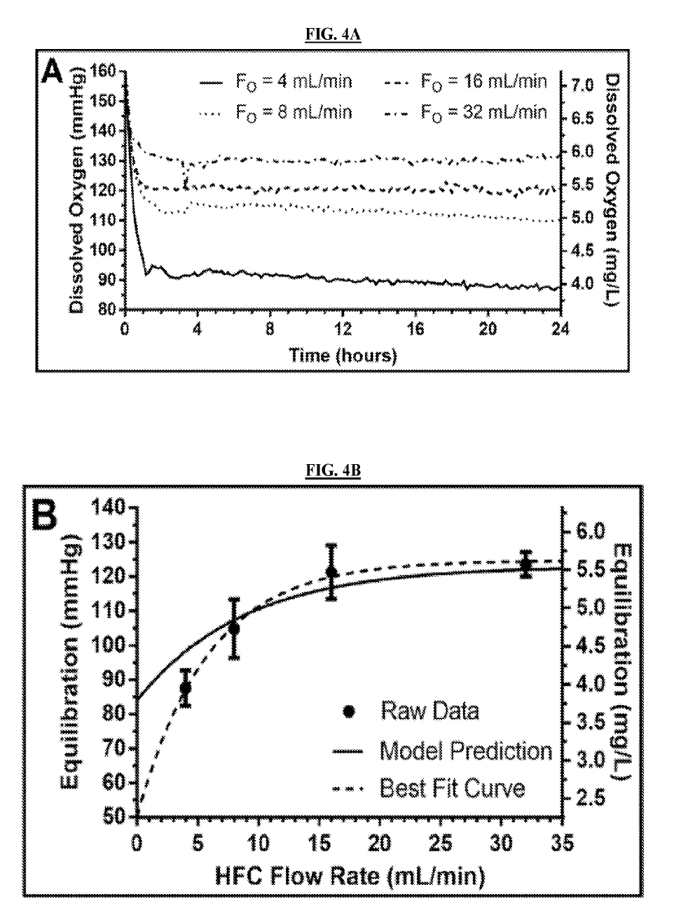

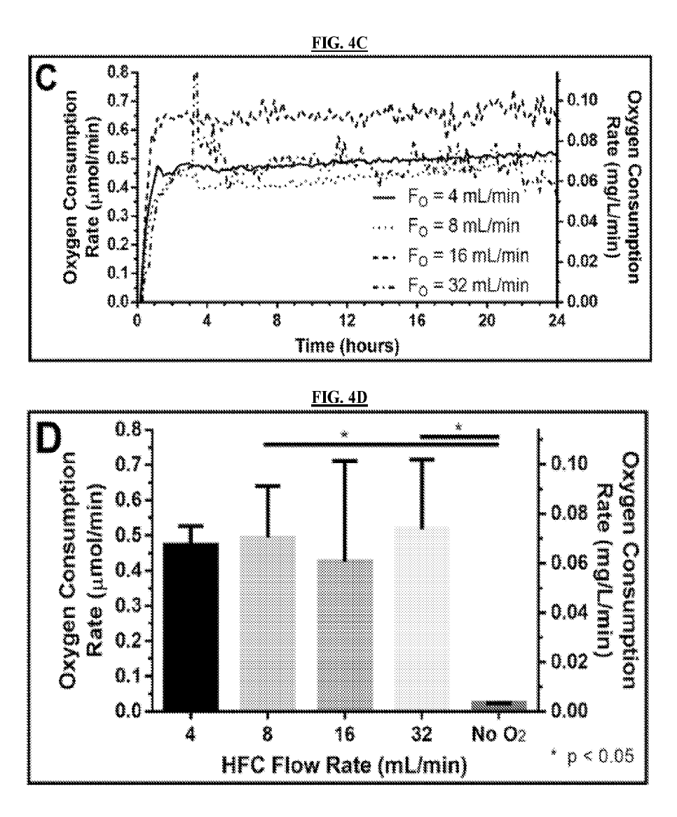

To assess the predictive value of this mathematical relationship, native lungs were cultured at four different rates of gas exchange flow for 24 hours. Representative dissolved oxygen traces are shown in FIG. 4A. The preparation time for these 12 lungs was 33±3 min, and the estimated whole lung cell count was 442±101 million cells. All four of the dissolved oxygen traces follow a similar trend, starting with an initial decrease followed by an equilibration within one to four hours, with slower HFC flow rates resulting in a lower pO⁰² at equilibrium.

FIG. 4B shows the equilibration values as a function of the HFC flow rate, with N=3 for each point. The model prediction from above is overlaid as a solid trace, and a best-fit curve calculated in MATLAB® through non-linear curve fitting is overlaid as a dashed line. The model prediction aligns well with the HFC flow rates of 8, 16, and 32 mL/min, but the actual equilibration values at 4 mL/min are lower than predicted. This may be caused by technical factors: small bubbles become trapped in the hollow fiber cartridge at slow flow rates, thereby lowering the surface area for gas exchange.

Given these findings, the constants in the mathematical model were updated to best reflect the actual behavior of the oxygenator. B was assumed to be the least flexible variable, as FIG. 2C demonstrates tight clustering at high HFC flow rates. As such, the value of B was maintained, and the values of S, A, and T were updated to yield an updated Equation 3:

C_{B}=(5.997 mg/L)−(\dot{Q}_{N}/0.225 L)·((51.85 min)·exp(−F_{O}/(5.829 mL/min))+(5.765 min))

The equation above provides a quantitative relationship between the dissolved oxygen, the gas exchange flow rate, and the oxygen consumption rate of the whole lung. With this relationship, the dissolved oxygen data in FIG. 4A were transformed into real-time whole lung oxygen consumption rate data (FIG. 4C). This demonstrates the initial acclimation of the organ to its new environment after explanation from the donor, and the subsequent increase in oxygen consumption after approximately an hour of bioreactor culture. The oxygen consumption rates for all twelve lungs are shown in FIG. 4D as a function of the HFC flow rate, compared to the final oxygen consumption rate for the lungs cultured without oxygen (N=3 for all bars). There is no statistically significant difference between the four HFC flow rates (p=0.9429), and increases in oxygen consumption rate as compared to the “No O₂” culture (p=0.0552, 0.0439, 0.0918, 0.0331; No O₂ vs 4, 8, 16, and 32, respectively). This suggests that the culture system is able to support similar levels of whole organ oxygen consumption regardless of the pO₂ during culture, within this tested experimental range.

### Example 4: Immunofluorescence

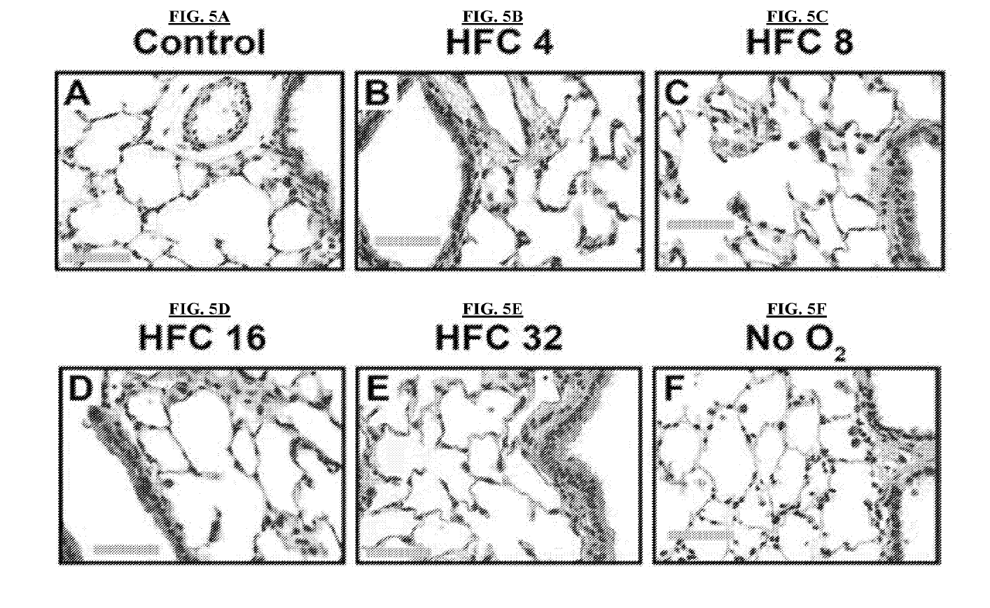

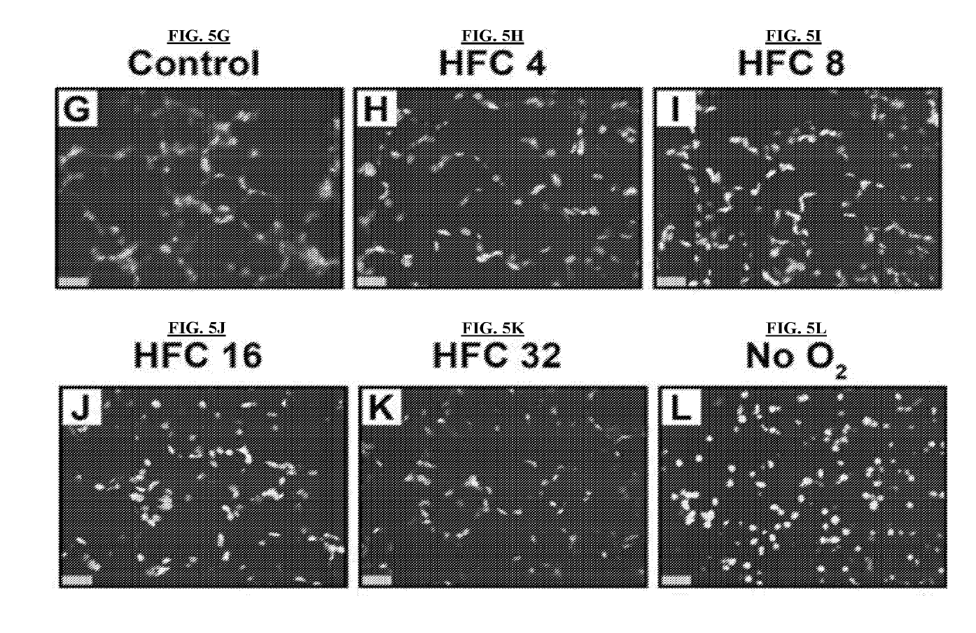

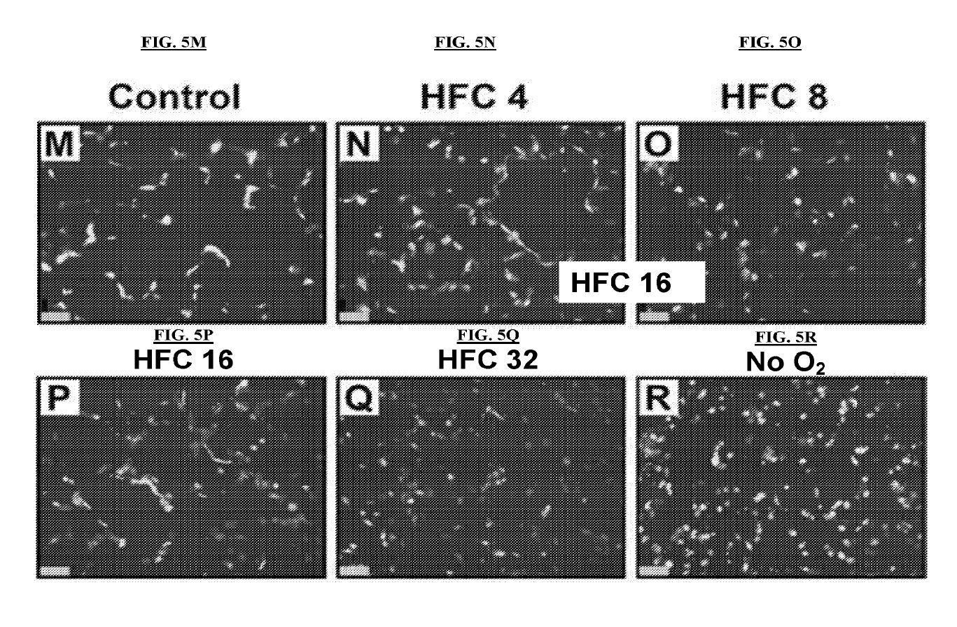

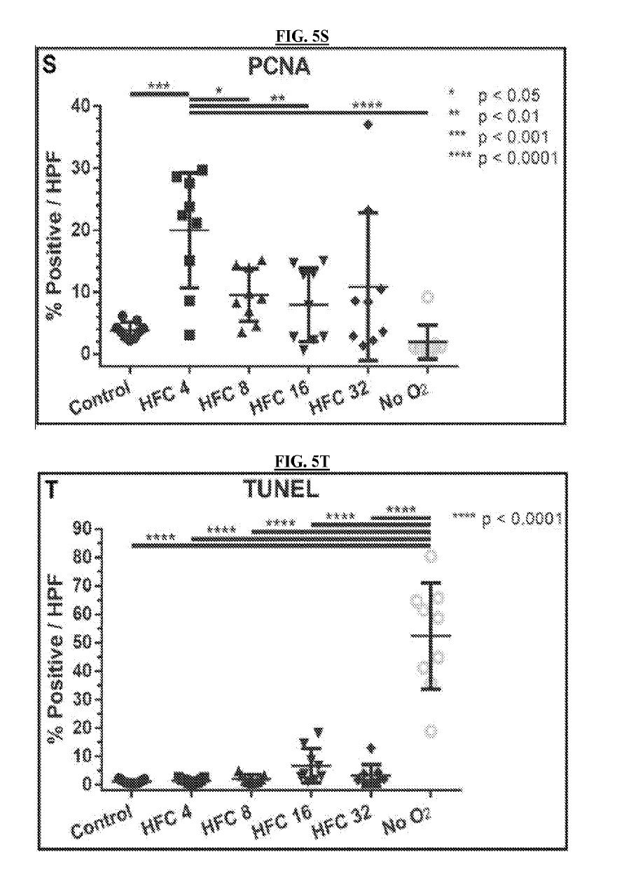

Histological images were taken for control lungs (freshly excised from rats), for lungs maintained at each of the four HFC flow rates, and for lungs cultured without oxygen (“No O₂”) (FIG. 5A-F). Histology shows all HFC flow rates maintain gross cell phenotype in both distal and proximal regions, while the No O₂ culture displays rounding of cell nuclei and detachment of cells from the extracellular matrix, demonstrating the adverse effects of extreme hypoxia.

Representative PCNA and TUNEL images are also shown (FIG. 5G-R), along with quantification of positive nuclei for each stain (FIG. 5S-T). Images contained 167±33 nuclei (mean+SD). Lungs cultured at HFC flow rates of 4 mL/min demonstrated significantly higher rates of proliferation as compared to the other experimental groups, suggesting that mild hypoxia may trigger increased proliferation. While not significant, these data also show slight increases in the percentage of cells staining for PCNA for all lungs cultured under gas exchange as compared to the controls, and decreases in PCNA staining for the lungs cultured without oxygen as compared to the controls. TUNEL staining shows a highly statistically significant increase in apoptosis in the group lacking oxygen as compared to every other group, with no statistically significant differences between any of the HFC lungs and the controls. Taken together, these data support the idea that the bioreactor with the hollow fiber oxygenator is able to support whole lung viability.

### Example 5: Variable Correlations

The relationship between experimental variables was investigated through a series of one-way ANOVAs and linear regressions to determine significant correlations, with a complete listing of R² and p values reported in Table 2. The HFC flow rate only had statistically significant correlations with the equilibrium pO₂, highlighting that the pO₂ values explored in these experiments had no significant effect on nutrient utilization. The preparation time for the lungs used in these experiments only had statistically significant correlations with TUNEL staining. This correlation emphasizes the exquisite sensitivity of lung tissues to periods of ischemia, as the addition of a few minutes of prep time yields a significant increase in the percentage of cells staining positively for apoptotic markers.

Rat weight and lung weight have statistically significant correlations with whole-organ nutrient consumption. This relationship intuitively makes sense, as it suggests that heavier rats and larger lungs will consume more oxygen and glucose and will produce more lactate. However, neither rat nor lung weight show statistical significance at the single cell level for either oxygen, glucose, or lactate. This absence of significance could suggest that this bioreactor system is an effective tool for investigating metabolic activity of an average single lung cell in situ, regardless of organ size or cell number. The mean single cell oxygen consumption rate for these lungs was 1.20±0.59×10¹⁵ mol/min/cell, compared to previously reported values for lung epithelium at 1.12×10¹⁵ mol/min/cell. This would suggest that lung cells consume oxygen at roughly the same rate in their native environment as compared to isolated cells. However, the average values for single cell glucose consumption (0.402±0.244 ng/cell/day) and lactate production (0.335±0.163 ng/cell/day) were significantly lower than literature values for freshly isolated cells (glucose 0.912±0.061 ng/cell/day, lactate 1.129±0.108 ng/cell/day), suggesting that lung cells are less metabolically active in situ as compared to isolated, individual cells. These data also demonstrate no correlation between any variable to Y_{l/g}, the ratio of lactate consumption to glucose production and an indicator of overall metabolic state. Furthermore, the mean ratio of 0.78±0.13 is lower than literature values of 1.24±0.20 for freshly isolated lung cells. Not only do these findings suggest that heavier lungs behave metabolically similarly to lighter lungs, but also that lung cells in situ participate in proportionally more aerobic metabolism (where Y_{l/g}=0) than anaerobic metabolism (where Y_{l/g}=2) as compared to isolated cells.

TABLE 2

| | p value | | | | R² | | |
|---|---|---|---|---|---|---|---|
| | HFC | Rat | Lung | Prep | Rat | Lung | Prep |
| | Speed | Weight | Weight | Time | Weight | Weight | Time |
| Equilibrium pO₂ | 0.0005 | 0.4778 | 0.5811 | 0.5181 | 0.0516 | 0.0315 | 0.0429 |
| O₂ Consumption Rate | 0.9429 | 0.0511 | 0.0263 | 0.4258 | 0.3293 | 0.4041 | 0.0645 |
| Δ Glucose | 0.8064 | 0.0227 | 0.1657 | 0.4645 | 0.4200 | 0.1827 | 0.0547 |
| Δ Lactate | 0.7543 | 0.1177 | 0.0477 | 0.3571 | 0.2267 | 0.3374 | 0.0853 |
| Y_{I/g} | 0.3162 | 0.1522 | 0.7632 | 0.8725 | 0.1937 | 0.0095 | 0.0027 |
| Total Cell Count | 0.5723 | 0.5472 | 0.2124 | 0.2483 | 0.0374 | 0.1507 | 0.1307 |
| Single Cell O₂ | 0.9878 | 0.0991 | 0.1091 | 0.2440 | 0.2484 | 0.2362 | 0.1329 |
| Consumption Rate | | | | | | | |
| Single Cell | 0.9519 | 0.0566 | 0.2230 | 0.2719 | 0.3172 | 0.1444 | 0.1191 |
| Δ Glucose | | | | | | | |
| Single Cell | 0.9893 | 0.1511 | 0.1011 | 0.1662 | 0.1947 | 0.2458 | 0.1823 |
| Δ Lactate | | | | | | | |
| % PCNA | 0.2292 | 0.7572 | 0.7522 | 0.1646 | 0.0100 | 0.0104 | 0.1836 |
| % TUNEL | 0.1006 | 0.1538 | 0.8739 | 0.0153 | 0.1923 | 0.0026 | 0.4605 |

### Example 6: Self-Regulating Bioreactor Apparatus

Decellularized lung scaffolds were prepared from adult SPRAGUE DAWLEY® male rats using established methods (Calle, E. A., et al, Acta Biomater; Volume 46, December 2016, Pages 91-100; Balestrini, J. L., et al, Biomaterials; Volume 102, September 2016, Pages 220-230). Lung scaffolds were stored in an antibiotic/antimycotic solution (PBS with 1000 μg/mL penicillin, 1000 μg/mL streptomycin, 10 μg/mL amphotericin B, and 200 μg/mL gentamicin). 24 hours prior to cell seeding, lungs were perfused at 2 mL/min into the pulmonary artery using a peristaltic pump. 2 hours prior to cell seeding, lungs were transferred to a fresh bioreactor prepared with 225 mL of cell-specific culture medium and perfused at 2 mL/min with culture medium. This bioreactor used was the bioreactor described in Example 1—containing a perfusion loop to perfuse the lung via the pulmonary artery, an oxygenation loop to re-introduce gases into the bioreactor via a hollow fiber cartridge, and air filters—and additionally included a connection point for the trachea cannula to enable cell seeding into the alveolar compartment.

Two cell lines were chosen for investigation: Normal Human Bronchial Epithelial Cells (HBEs) and A549 cells, an adenocarcinomic human alveolar basal epithelial cell line. Cells were expanded on tissue culture plastic to achieve 25 million cells. HBEs were cultured in Minimum Essential Medium (MEM) with 10% Fetal Bovine Serum (FBS), 1% L-Glutamine, 100 U/mL penicillin, 100 μg/mL streptomycin, 3 μg/mL amphotericin B, and 50 μg/mL gentamicin. A549s were cultured in Dulbecco's Modified Eagle Medium (DMEM) High Glucose, 10% FBS, 100 U/mL penicillin, 100 μg/mL streptomycin, 3 μg/mL amphotericin B, and 50 μg/mL gentamicin. Prior to cell seeding, cells were trypsinized and counted, and 25 million cells were resuspended in 10 mL of media.

Cells were introduced into the alveolar compartment of the lung scaffold via the trachea. A syringe containing the cellular suspension with the plunger removed was connected in-line with the tracheal cannula. A vacuum of −5 mmHg was created in the bioreactor, passively drawing the cellular suspension into the alveolar compartment. The 10 mL of cells was followed by a 3 mL chaser of cell-free media, after which point the bioreactor was sealed off, still under vacuum. The lung was allowed to rest, still inflated, for either 1 hour (A549s) or 2 hours (HBEs) to allow for cellular attachment, after which point the air filters were uncapped and all pumps and sensors were turned on. The perfusion flow rate was gradually increased over 90 minutes to a final perfusion rate of 10 mL/min. The oxygenation flow rate through the hollow fiber cartridge was set at 10 mL/min for the entirety of culture. Dissolved oxygen data was collected every 10 seconds as previously described. Culture continued for 96 hours. Every 24 hours, perfusion was briefly stopped and a media change was performed, removing 200 mL of media and replacing it with 200 mL of fresh media.

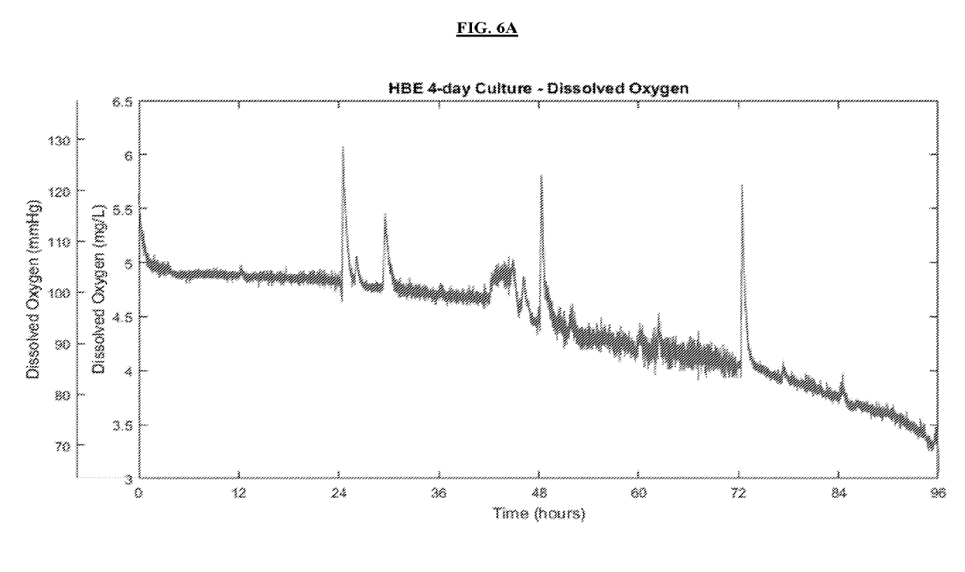

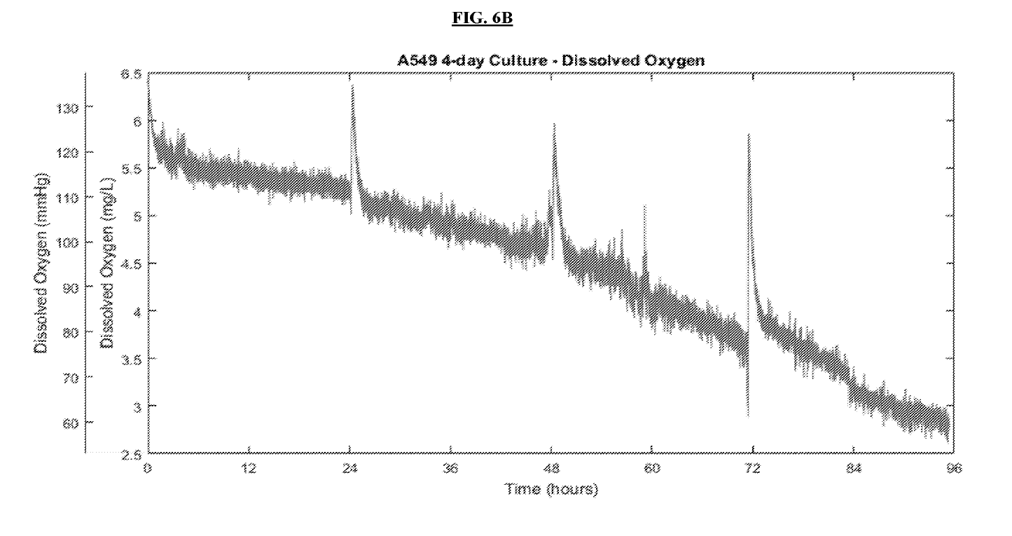

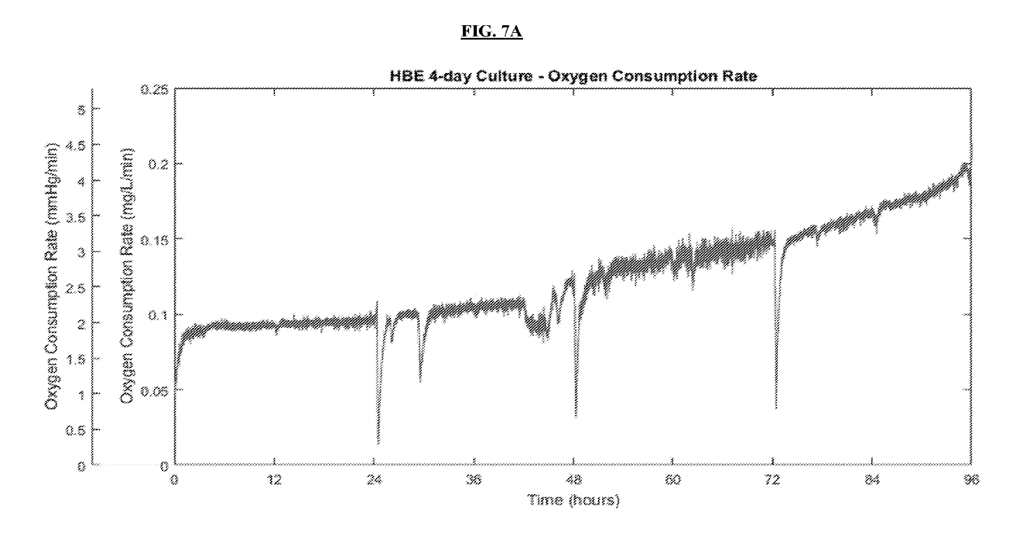

FIGS. 6A-6B show the dissolved oxygen data as a function of time, for both HBEs (FIG. 6A) and A549s (FIG. 6B). Both traces follow a similar pattern over the four day culture: (1) an initial equilibration as cells begin to utilize oxygen again, (2) a short steady state period where the concentration of dissolved oxygen is fairly constant for a few hours, and (3) a decrease in dissolved oxygen that begins slowly and increases in rate as culture continues. Media changes at 24, 48, and 72 hours result in oxygen readings transiently increasing (as the probe becomes exposed to air and then fresh media), followed by a subsequent return to normal levels. The culture with HBEs starts at a lower steady state concentration of oxygen than the A549s, but oxygen levels in the A549 culture decrease at a faster rate, resulting in a lower concentration of oxygen after four days of culture.

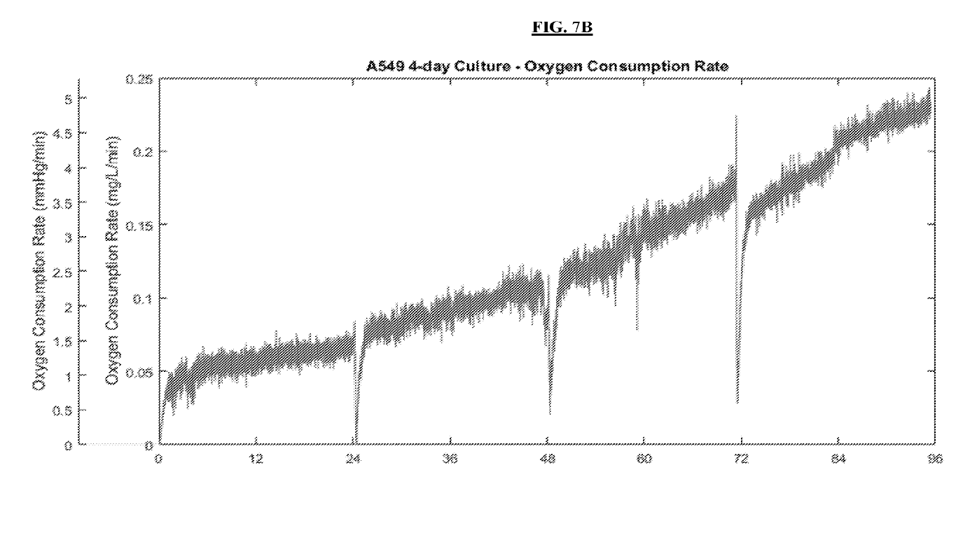

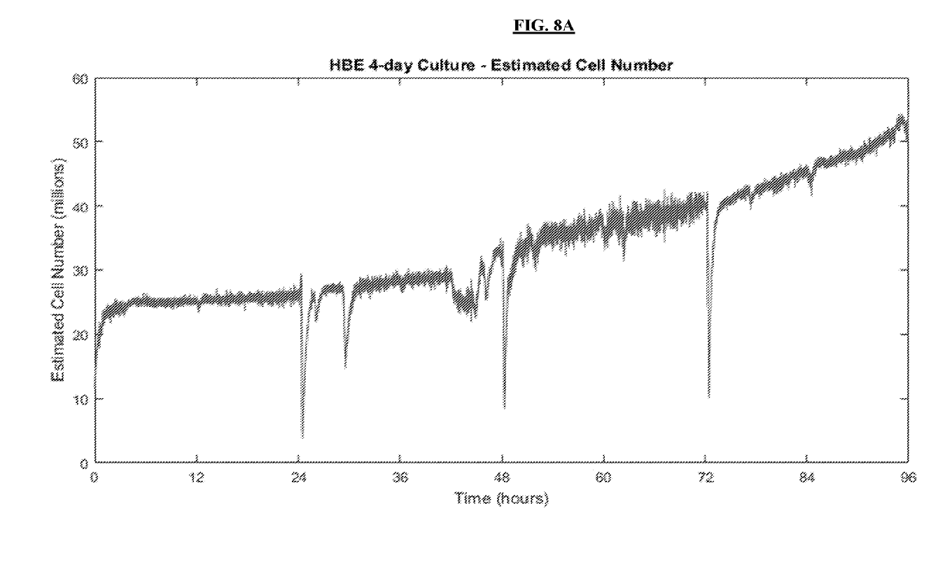

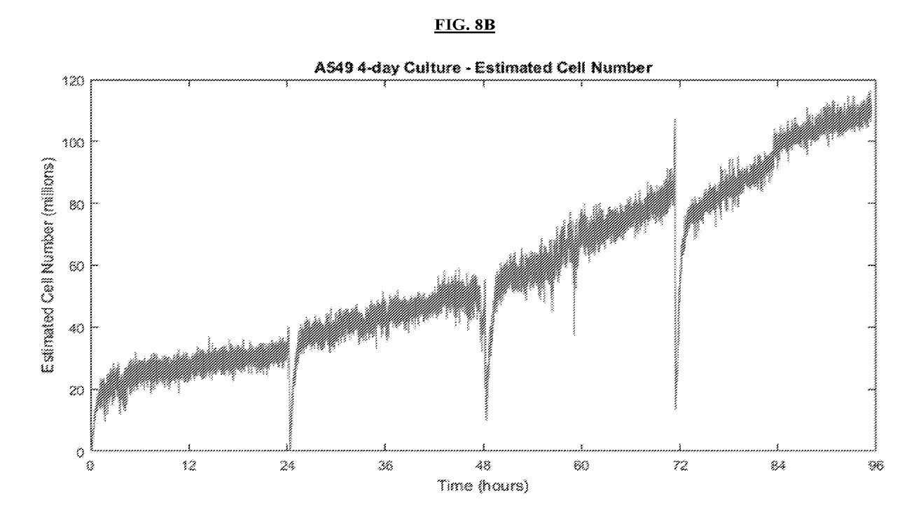

FIGS. 7A-7B show the calculated whole organ oxygen consumption rate as a function of time, for both HBEs (FIG. 7A) and A549s (FIG. 7B). These values were calculated using the mathematical model previously described, inputting the dissolved oxygen data from FIGS. 6A-6B and the oxygenation flow rate of 10 mL/min. Both curves show exponential-like growth in whole organ oxygen consumption rate. The culture with HBEs begins with a higher whole organ oxygen consumption rate than the A549s, but the whole organ oxygen consumption rate in the A549 culture increases at a faster rate, resulting in a higher whole organ oxygen consumption rate after four days of culture.

These whole organ oxygen consumption rate data were then transformed into estimated cell number using the equation

$${N}_{n} = \frac{{\dot{Q}}_{n} \cdot {N}_{0}}{{\dot{Q}}_{0}} .$$

The initial cell number of 25 million cells (N₀) was assigned to the steady-state whole-oxygen consumption rate around time t=4 hours ( [symbol] ₀). Using [symbol] the formula above, the real-time estimated cell number for both HBEs and A549s are shown in FIG. 8A and FIG. 8B, respectively. This representation of the data shows exponential-like growth for both cell types, with A549s growing at a markedly faster rate than the HBEs.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A device for quantifying and controlling oxygen concentration within a bioreactor containing a cell-containing sample that is actively consuming oxygen, the device comprising:
a bioreactor vessel adapted and configured to receive a cell-containing sample;
a perfusion loop adapted and configured to circulate a perfusate from within the bioreactor vessel and back into the bioreactor vessel, the perfusion loop comprising a first pump;
a gas exchanger comprising one or more gas exchange sources adapted and configured to add or remove gases from the perfusate;
a sensor within the bioreactor adapted and configured to measure the dissolved oxygen concentration in the perfusate; and
a controller programmed to control one or more parameters selected from the group consisting of the specified flow rate of the perfusate through the gas exchanger and the rate of gas exchange through the one or more gas exchange sources.

2. The device of claim 1, wherein the device only exchanges gases through the one or more gas exchange sources and is otherwise substantially sealed off from the ambient atmosphere.

3. The device of claim 1, wherein the gas exchanger is integrated in-line into the perfusion loop and wherein the controller is further programmed to modulate the specified flow rate of gas exchange through the one or more gas exchange sources.

4. The device of claim 1, wherein the perfusion loop is adapted and configured to circulate a perfusate from within the bioreactor vessel, through the cell-containing sample and back into the bioreactor vessel.

5. The device of claim 1, wherein the gas exchange sources are adapted and configured to introduce oxygen into or remove oxygen from the perfusate.

6. The device of claim 1, wherein the controller is further programmed to calculate an oxygen consumption rate for the cell-containing sample.

7. The device of claim 6, wherein the controller is programmed to calculate an oxygen consumption rate for the cell-containing sample utilizing a differential equation relating:
instantaneous oxygen consumption rate;
dissolved oxygen concentration in the perfusate; and
known system parameters derived for the specific device configuration.

8. The device of claim 1, wherein the controller is further programmed to maintain a steady oxygen concentration in the perfusate in the event of a change in oxygen concentration.

9. The device of claim 8, wherein the change in oxygen concentration in the perfusate is due to a change in oxygen consumption.

10. The device of claim 9, wherein the controller is programmed to maintain a steady oxygen concentration in the perfusate by:
calculating an oxygen consumption rate for the cell-containing sample utilizing a differential equation relating:
instantaneous oxygen consumption rate;
dissolved oxygen concentration in the perfusate; and
known system parameters derived for the specific device configuration; and
altering the system parameters in order to maintain a steady oxygen concentration in the perfusate.

11. The device of claim 8, wherein the change in oxygen consumption is due to cell proliferation, cell degradation or metabolic shift within the cell-containing sample.

12. The device of claim 1, wherein the oxygen consumption rate can be determined without sealing the system from the one or more gas exchange sources and wherein oxygen consumption can be tracked continuously in real time.

13. The device of claim 1, wherein the sensor is selected from the group consisting of: an optical dissolved oxygen probe and a dissolved oxygen electrode.

14. The device of claim 1, wherein the cell-containing sample is selected from the group consisting of: a cell culture, a tissue segment, a partial organ, a whole organ and an organ mimic.

15. The device of claim 14, wherein the cell culture is a culture comprising at least one selected from the group consisting of: adherent cells, cells suspended in a fluid, cells suspended in a gel and a self-assembling cellular scaffold.

16. The device of claim 1, wherein the cell-containing sample comprises tissue from one or more organs selected from lung, heart, kidney, liver, vessel, trachea, skin, pancreas, bladder, cartilage and bone.

17. The device of claim 1, wherein the cell-containing sample is derived from a source selected from the group consisting of murine, canine, ovine, porcine, bovine and primate sources.

18. The device of claim 1, wherein the cell-containing sample is derived from a human.

19. The device of claim 1, wherein the perfusate comprises a phosphate-buffered saline solution.

20. The device of claim 1, wherein the perfusate comprises a culture medium containing one or more cellular growth factors and/or one or more nutrients.

21. The device of claim 1, wherein the one or more gas exchange sources are hollow fiber supported membranes that are exposed to a gas source.

22. The device of claim 21, wherein the supported membranes comprise one or more materials selected from the group consisting of polydimethylsiloxane, polymethylpentene, polyethersulfone and polysulfone.

23. The device of claim 21, wherein the gas source is an oxygen source.

24. The device of claim 23, wherein the gas source comprises at least about 0.001% oxygen by volume.

25. The device of claim 1, wherein the controller is further programmed to collect oxygen concentration levels and flow rates about every 500 milliseconds to about every 1 hour.

26. The device of claim 1, wherein the main body of the bioreactor comprises a vessel comprising one or more materials selected from the group consisting of stainless steels, borosilicates, platinum-cured silicones, polysulfones, fluoropolymers, polyethylenes and acrylics.

27. The device of claim 1, wherein the perfusate within the bioreactor is stirred.

28. The device of claim 1, wherein the gas exchanger is a gas exchange loop adapted and configured to circulate the perfusate in the bioreactor vessel through the one or more gas exchange sources and back into the bioreactor vessel, the gas exchange loop further comprising a second pump that is controllable to operate at a specified fluid flow rate.

29. The device of claim 28, wherein the rate of gas exchange through the one or more gas exchange sources is constant.

30. The device of claim 28, wherein the controller is programmed to control the specified flow rate of the perfusate through the gas exchange loop.

31. The device of claim 30, wherein the controller is programmed to control the gas flow rate through the one or more gas exchange sources.

32. The device of claim 28, wherein the controller is programmed to calculate an oxygen consumption rate for the cell-containing sample by:
receiving a dissolved oxygen concentration C_{B} value from the sensor;
measuring a flow rate F_{O} for the gas exchange loop and a flow rate Fₚ for the perfusion loop; and
solving the differential equation Ċ_{B}=F_{O} (C_{O}−C_{B})−F_{P} (C_{B}−C_{L}), wherein:
C_{O} is a concentration of oxygen leaving the gas exchange sources; and
C_{L} is a concentration of oxygen leaving the cell-containing sample.

33. The device of claim 28, wherein the controller is programmed to calculate an oxygen consumption rate for the cell-containing sample by:
receiving a dissolved oxygen concentration C_{B} value from the sensor;
measuring a flow rate F_{O} for the gas exchange loop; and
solving the equation C_{B}=S(F_{O})−\dot{Q}₀·τ(F_{O})/V for oxygen consumption rate \dot{Q}₀, wherein:
S(F_{O}) is an experimentally-determined system saturation function of F_{O};
τ(F_{O}) is an experimentally-determined system time constant as a function of F_{O}; and
V is a total amount of fluid volume in the bioreactor, perfusion loop, and gas exchange loop.

34. The device of claim 33, wherein the controller is further programmed to calculate an estimated average single cell oxygen consumption rate
$$\frac{{Q}_{0}}{{N}_{0}}$$
for a tissue sample comprising an initial known number of cells N₀.

35. The device of claim 28, wherein the controller is programmed to maintain a steady oxygen concentration in the perfusate through self-regulation by:
measuring oxygen concentration C_{B} from within the bioreactor;
measuring a flow rate F_{O} for the gas exchange loop;
solving the equation C_{B}=S(F_{O})−\dot{Q}₀·τ(F_{O})/V for oxygen consumption rate \dot{Q}₀, wherein:
S(F_{O}) is an experimentally-determined system saturation function of F_{O};
τ(F_{O}) is an experimentally-determined system time constant as a function of F_{O}; and
V is a total amount of fluid volume in the bioreactor, perfusion loop, and gas exchange loop; and
adjusting F_{O} in order to maintain a steady C_{B} value.

36. A method of non-invasively estimating changes in a number of cells within a cell-containing sample using the device of claim 28, the method comprising:
measuring oxygen concentration C_{B} from within the bioreactor;
measuring a flow rate F_{O} for the gas exchange loop;
solving the equation C_{B}=S(F_{O})−\dot{Q}₀·τ(F_{O})/V for oxygen consumption rate \dot{Q}₀ at an initial condition in which the cell-containing sample has a known number of cells N₀, wherein:
S(F_{O}) is an experimentally-determined system saturation function of F_{O};
τ(F_{O}) is an experimentally-determined system time constant as a function of F_{O}; and
V is a total amount of fluid volume in the bioreactor, perfusion loop, and gas exchange loop; and
solving the equation C_{B}=S(F_{O})−\dot{Q}ₙ·τ(F_{O})/V for oxygen consumption rate \dot{Q}ₙ for a later condition in which the cell-containing sample has an unknown number of cells Nₙ; and
solving the equation
$${N}_{n} = \frac{{\dot{Q}}_{n} \cdot {N}_{0}}{{\dot{Q}}_{0}} .$$

37. The device of claim 1, further comprising at least one sensor for measuring the concentration of at least one compound in the perfusate selected from the group consisting of: glucose, lactate, glutamate, glutamine and ammonia.

38. A method of non-invasively estimating metabolic activity in a cell-containing sample using the device of claim 37, the method comprising:
measuring a change in glucose ΔGₙ and a change in lactate ΔLₙ in the perfusate over a period of time under an initial condition;
solving the equation % A₀=(2−ΔLₙ/ΔGₙ)/2 to determine the portion of cells participating in aerobic metabolism % A₀ under the initial condition; and
solving the equation \dot{Q}₀=\dot{Q}_{1A}*% A₀*N₀ for single cell aerobic oxygen consumption rate \dot{Q}_{1A} at the initial condition in which the cell-containing sample has a known number of cells N₀.

39. A method of non-invasively estimating changes in a number of cells within a cell-containing sample using the device of claim 37, wherein fewer than 100% of cells are participating in aerobic metabolism, the method comprising:
measuring a change in glucose ΔGₙ and a change in lactate ΔLₙ in the perfusate over a period of time under an initial condition;
solving the equation % A₀=(2−ΔLₙ/ΔGₙ)/2 to determine the portion of cells participating in aerobic metabolism % A₀ under the initial condition;
solving the equation \dot{Q}₀=\dot{Q}_{1A}*% A₀*N₀ for single cell aerobic oxygen consumption rate \dot{Q}_{1A} at the initial condition in which the cell-containing sample has a known number of cells N₀; and
calculating a portion of cells participating in aerobic metabolism during a later condition by a further method comprising:
measuring a change in glucose ΔGₙ and a change in lactate ΔLₙ in the perfusate over a period of time during a culture period;
solving an equation % Aₙ=(2−ΔLₙ/ΔGₙ)/2 to determine a portion of cells participating in aerobic metabolism % Aₙ; and
solving the equation
$$\frac{{\dot{Q}}_{0}}{\% {A}_{0} \cdot {N}_{0}} = \frac{{\dot{Q}}_{n}}{\% {A}_{n} \cdot {N}_{n}}$$
for Nₙ, an unknown number of cells in the cell-containing sample during the later condition.
